(19) United States
(12) Patent Application Publication
(10) Pub. No.: US 2024/0325546 A1
(43) Pub. Date: Oct. 3, 2024

(54) TOPICAL DELIVERY OF THERAPEUTIC OLIGONUCLEOTIDES

(71) Applicants: UNIVERSITY OF MASSACHUSETTS, Boston, MA (US); MCGILL UNIVERSITY, Montréal (CA)

(72) Inventors: Hassan H. Fakih, Montréal (CA); Hanadi Sleiman, Montréal (CA); Anastasia Khvorova, Westborough, MA (US); Qi Tang, Worcester, MA (US); John E. Harris, Sterling, MA (US); Julia Alterman, Westborough, MA (US); Mohammad Zain Ul Abideen, Boston, MA (US); Raymond Furgal, Oxford, MA (US)

(21) Appl. No.: 18/593,212

(22) Filed: Mar. 1, 2024

Related U.S. Application Data
(60) Provisional application No. 63/449,875, filed on Mar. 3, 2023.

(51) Int. Cl. A61K 47/54; A61M 37/00; C12N 15/113

(52) U.S. Cl. CPC A61K 47/549; A61K 47/542; A61M 37/0015; C12N 15/113; A61M 2037/0023; A61M 2037/0046; A61M 2037/0061; C12N 2310/14; C12N 2310/351

(57) ABSTRACT

This disclosure provides compositions, systems, and methods for the topical delivery of therapeutic oligonucleotides with microneedle particles.

### CROSS REFERENCE TO RELATED APPLICATION

This application claims the benefit of U.S. Provisional Patent Application Ser. No. 63/449,875, filed Mar. 3, 2023. The entire contents of the above-referenced patent application is incorporated by reference in their entirety herein.

### TECHNICAL FIELD

This disclosure relates to topical delivery of therapeutic oligonucleotides. In particular, provided herein are compositions, systems, and methods for topical delivery of oligonucleotide conjugates with microneedle particles.

### BACKGROUND

The utility of therapeutic oligonucleotides is limited in the ability of delivering said oligonucleotides to the target tissues and cells of interest. Topical delivery in particular is challenging with the skin acting as a robust barrier. Microneedling may be used to delivery compounds through the skin, but to date, microneedling has not been employed for the delivery of large therapeutics such as oligonucleotides.

Therefore, there remains a need for novel compositions, systems, and methods for the effective topical delivery of therapeutic oligonucleotides.

### SUMMARY

Provided herein are compositions, systems, and methods for the topical delivery of an oligonucleotide conjugate or branched oligonucleotide. The oligonucleotide conjugates or branched oligonucleotides can efficiently knockdown genes in target tissues. Several different oligonucleotide conjugates comprising different oligonucleotides and dendron demonstrated skin delivery upon administration.

In one aspect, the disclosure provides a method for topical delivery of an oligonucleotide conjugate or branched oligonucleotide to a subject, the method comprising: a) contacting a region of skin of the subject with a plurality of microneedle particles; and b) contacting the region of skin from step a) with the oligonucleotide conjugate or branched oligonucleotide, or contacting a region of skin of the subject with a topical formulation comprising a plurality of microneedle particles, an oligonucleotide conjugate or branched oligonucleotide, optionally a pharmaceutically acceptable carrier, and optionally a pharmaceutically acceptable salt, wherein the oligonucleotide conjugate comprises: i) an oligonucleotide comprising a 5′ and a 3′ end and complementarity to a target nucleic acid; and ii) a functional moiety linked to the oligonucleotide.

In certain embodiments, the microneedle particle comprises a core structure and one or more microneedles extending from the core structure.

In certain embodiments, the one or more microneedles is structured to penetrate a biological tissue.

In certain embodiments, at least one of (i) the core structure, (ii) the one or more microneedles, and (iii) a spatial relationship between or among two or more of the microneedles is configured to prevent the entire microneedle particle from penetrating the biological tissue.

In certain embodiments, the microneedle particle comprises three microneedles, four microneedles, five microneedles, six microneedles, seven microneedles, eight microneedles, or ten microneedles extending from the core structure.

In certain embodiments, the one or more microneedles are planar microneedles.

In certain embodiments, the microneedle particle comprises three or more microneedles, and at least one of the three or more microneedles is a non-planar microneedle.

In certain embodiments, each of the one or more microneedles independently has a length of about 10 μm to about 2,000 μm. In certain embodiments, each of the one or more microneedles independently has a length of about 100 μm to about 1,000 μm. In certain embodiments, each of the one or more microneedles independently has a length of about 100 μm to about 500 μm. In certain embodiments, at least one of the one or more microneedles is barbed.

In certain embodiments, upon penetrating the biological tissue at least once, at least one of the one or more microneedles is configured to fail mechanically, thereby preventing the at least one of the one or more microneedles from re-penetrating the biological tissue.

In certain embodiments, upon penetrating the biological tissue at least once, at least one of the one or more microneedles is configured to fail chemically, thereby preventing the at least one of the one or more microneedles from re-penetrating the biological tissue.

In certain embodiments, the microneedle particle comprises a metal, a polymer, a sugar, a sugar alcohol, or a combination thereof.

In certain embodiments, the microneedle particle has a matrix structure and a substance of interest dispersed in the matrix structure.

In certain embodiments, the matrix structure comprises a water-soluble or bioerodible material.

In certain embodiments, the microneedle particle is at least partially coated with a coating composition comprising a substance of interest.

In certain embodiments, the microneedle particle is formed of a substance of interest.

In certain embodiments, the functional moiety comprises any one of a dendron, retinoic acid, docosahexaenoic acid (DHA), docosanoic acid (DCA), and a-tocopheryl succinate.

In certain embodiments, the dendron comprises a hydrophilic end group, a phosphate group, and/or a hydrophobic chain.

In certain embodiments, the end group is a hydrophilic group comprising hydroxide, amine, phosphate, sulfur, and/or sugar; an hydrophobic group comprising amine, amide, ether, ester, N- or O-containing heterocycles, thiol, thioether, and/or saturated or unsaturated C₁₋₂₄ alkyl chain; and/or one or more aromatic rings.

In certain embodiments, the hydrophobic chain is a saturated or unsaturated C₁₋₂₄ alkyl group.

In certain embodiments, the dendron comprises two branches. In certain embodiments, the dendron comprises four branches. In certain embodiments, the dendron comprises eight branches.

In certain embodiments, the oligonucleotide conjugate has a structure of Formula I:

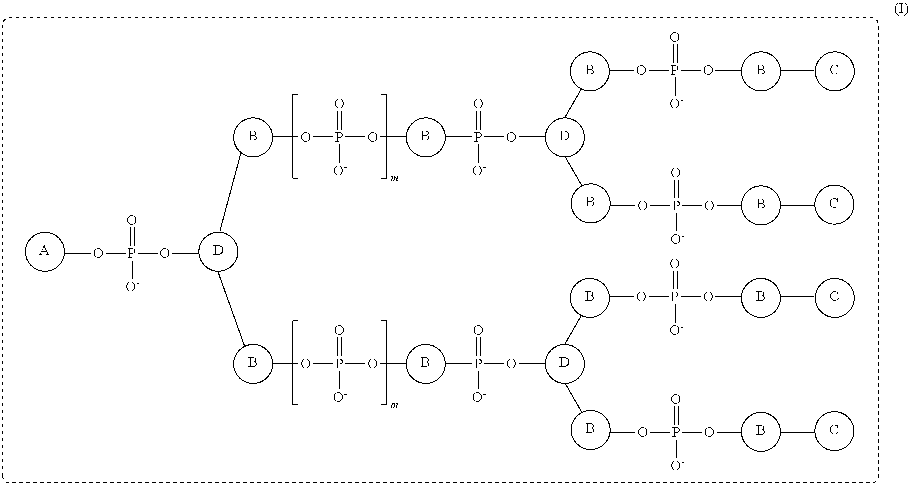

wherein: A is an oligonucleotide; B, for each occurrence, independently comprises one or more hydrophobic chains, amines, amides, esters, an N -or O-containing heterocycles, thioethers, disulfides, and/or aromatic rings, wherein the hydrophobic chain comprises a saturated or unsaturated C₁₋₂₄ alkyl chain; C, for each occurrence, independently comprises: an hydrophilic group comprising hydroxide, amine, phosphate, sulfur, and/or sugar; an hydrophobic group comprising amine, amide, ether, ester, N -or O-containing heterocycles, thiol, thioether, and/or saturated or unsaturated C₁₋₂₄ alkyl chain; and/or one or more aromatic rings; D, for each occurrence, independently is a branching unit comprising one or more alkyl chains, amides, ethers, esters, and amines, wherein the branching unit comprises 2 to 4 branches; and m, for each occurrence, independently is 0 or 1.

In certain embodiments, the oligonucleotide conjugate has a structure of Formula II:

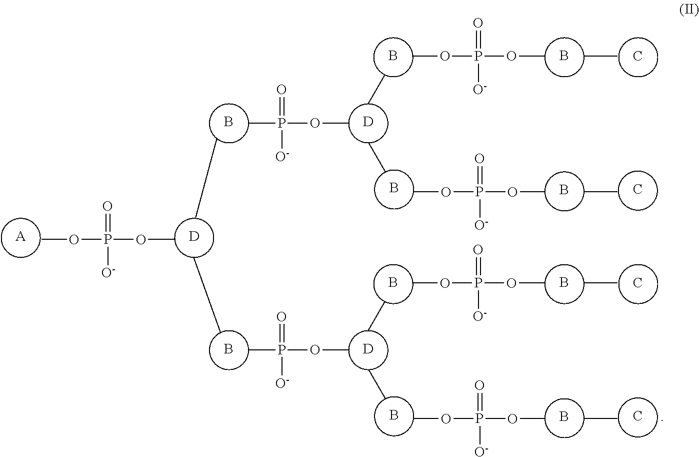

In certain embodiments, the oligonucleotide conjugate has a structure of Formula II:

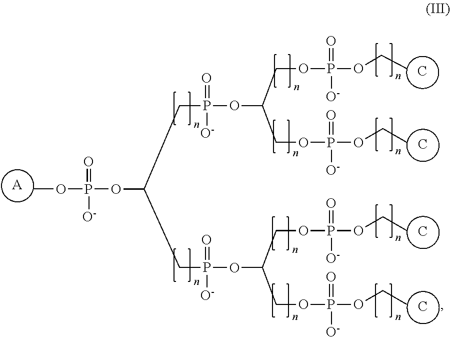

wherein n, for each occurrence, independently is an integer between 1-24.

In certain embodiments, C is OH and n, for each occurrence, independently is 1, 6, or 12.

In certain embodiments. the oligonucleotide coniugate has the structure of Formula IV:

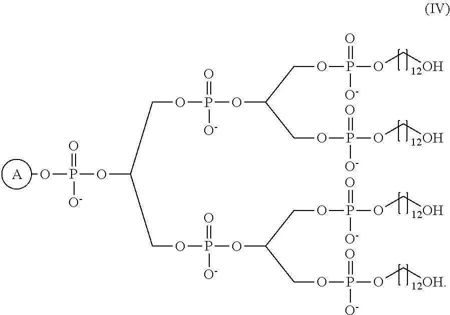

In certain embodiments, the oligonucleotide conjugate has a structure of Formula V:

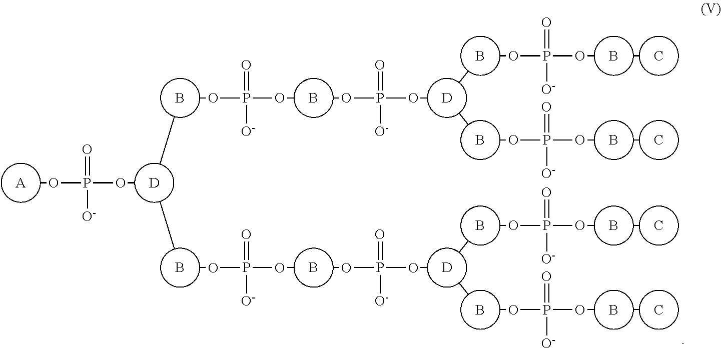

In certain embodiments, the oligonucleotide conjugate has a structure of Formula VI:

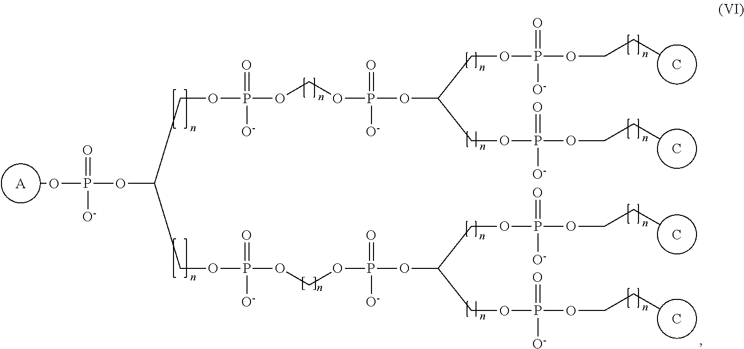

wherein n, for each occurrence, independently is an integer between 1-24.

In certain embodiments, C is OH and n, for each occurrence, independently is 1, 6, or 12.

In certain embodiments, the oligonucleotide conjugate has the structure of Formula VII

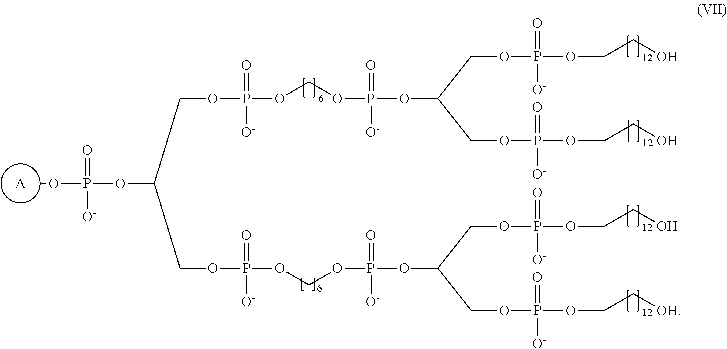

In certain embodiments, the functional moiety is linked to the 5′ end and/or 3′ end of the oligonucleotide.

In certain embodiments, oligonucleotide corresponds to an antisense oligonucleotide or a siRNA.

In certain embodiments, the siRNA comprises a sense strand and an antisense strand.

In certain embodiments, the functional moiety is linked to the 5′ end and/or 3′ end of the sense strand or to the 5′ end and/or 3′ end of the antisense strand.

In certain embodiments, the functional moiety is linked to the 3′ end of the sense strand.

In certain embodiments, the functional moiety is linked to the oligonucleotide (e.g., the antisense strand and/or sense strand) by a linker.

In certain embodiments, the linker comprises a divalent or trivalent linker.

In certain embodiments, the divalent or trivalent linker is selected from the group consisting of:

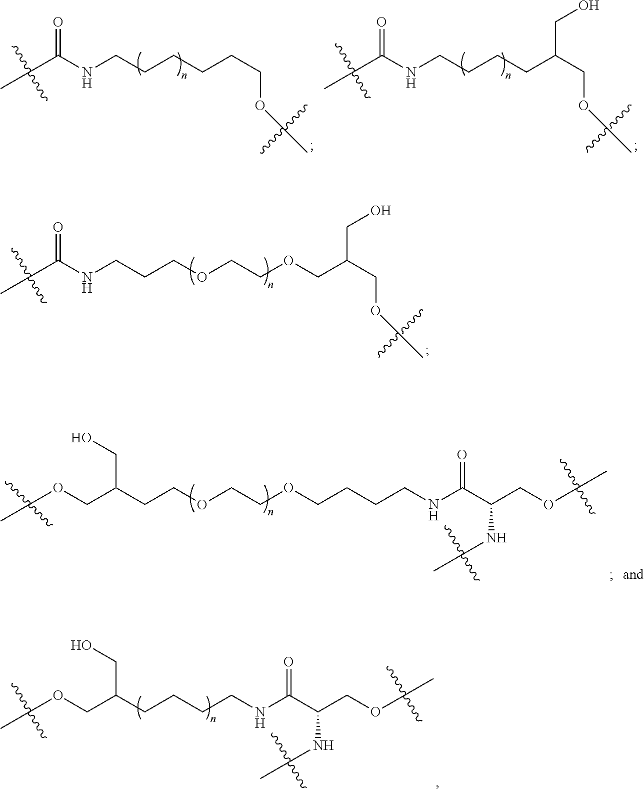

wherein n is 1, 2, 3, 4, or 5.

In certain embodiments, the linker comprises an ethylene glycol chain, an alkyl chain, a peptide, an RNA, a DNA, a phosphodiester, a phosphorothioate, a phosphoramidate, an amide, a carbamate, or a combination thereof.

In certain embodiments, when the linker is a trivalent linker, the linker further links a phosphodiester or phosphodiester derivative.

In certain embodiments, the phosphodiester or phosphodiester derivative is selected from

the group consisting of:

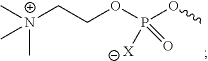

### (Zc1);

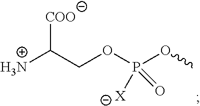

### (Zc2);

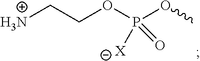

### and (Zc3)

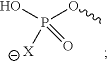

(Zc4), wherein X is O, S or BH₃.

In certain embodiments, the oligonucleotide conjugate comprises the structure:

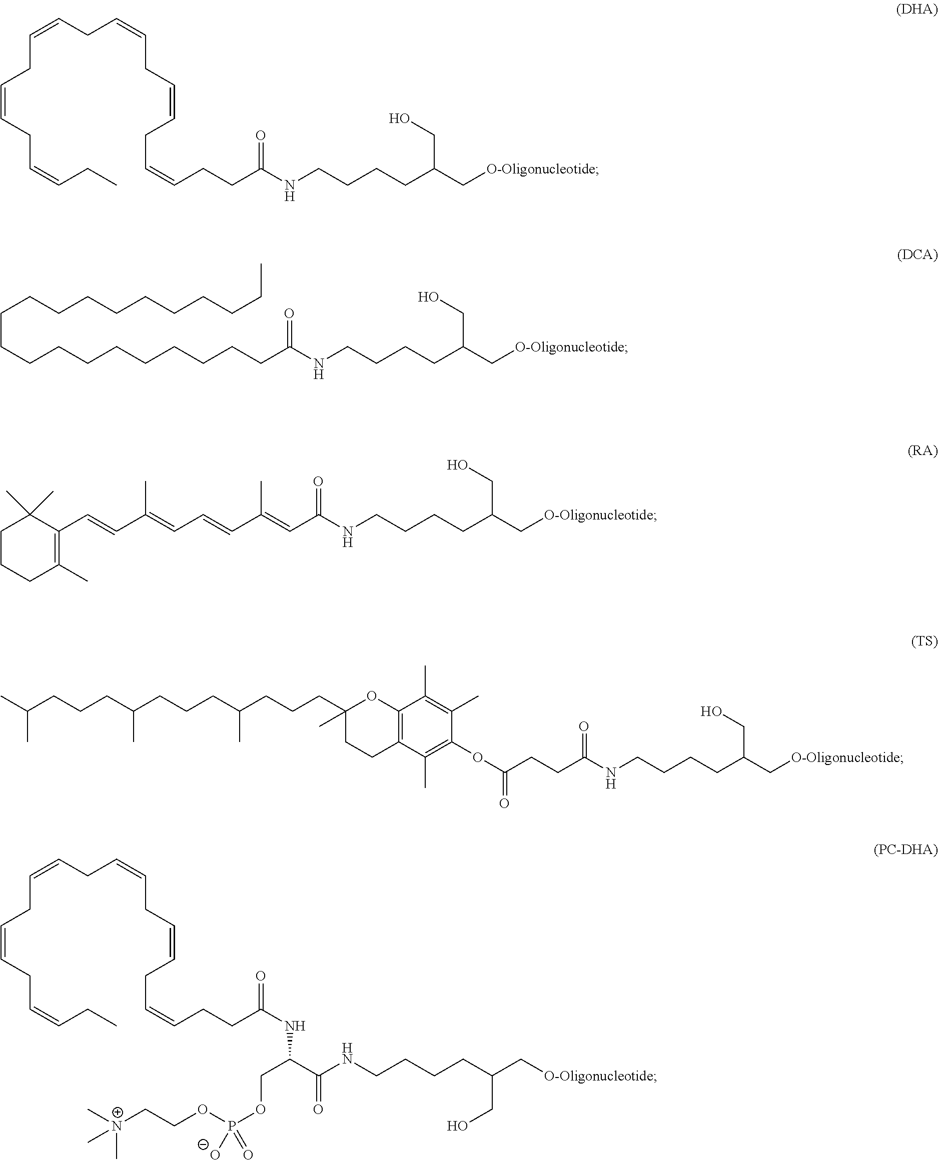

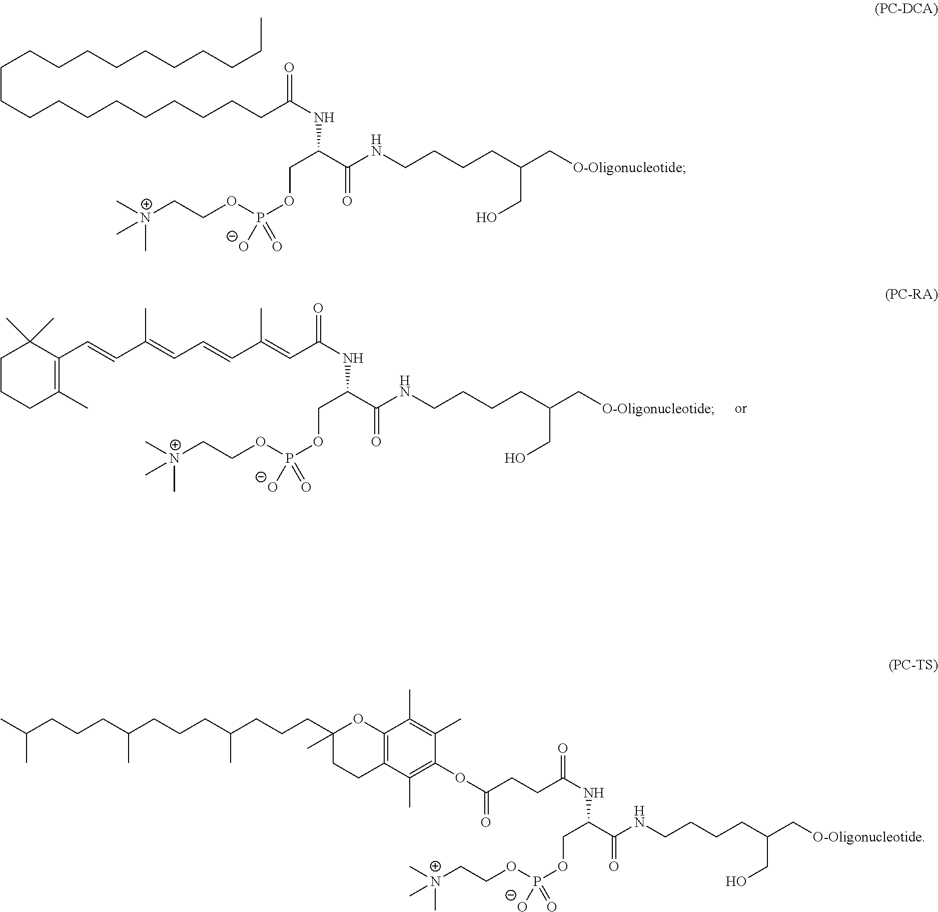

In certain embodiments, the branched oligonucleotide comprises the structure:

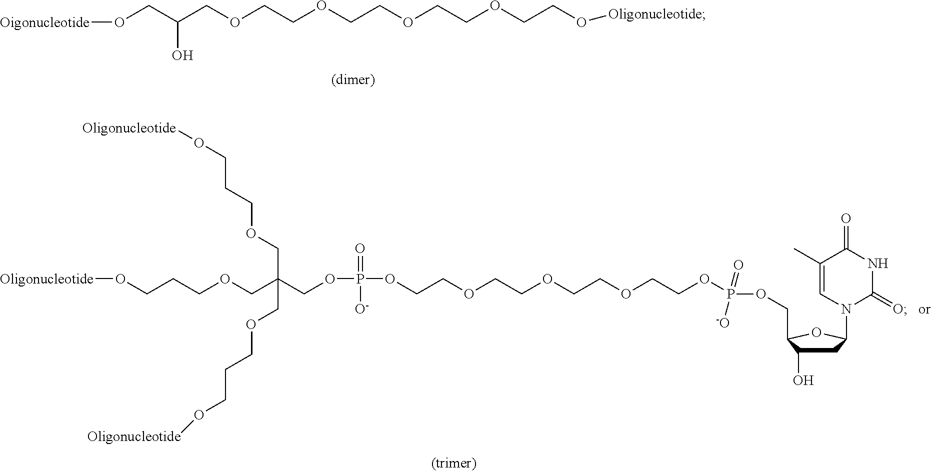

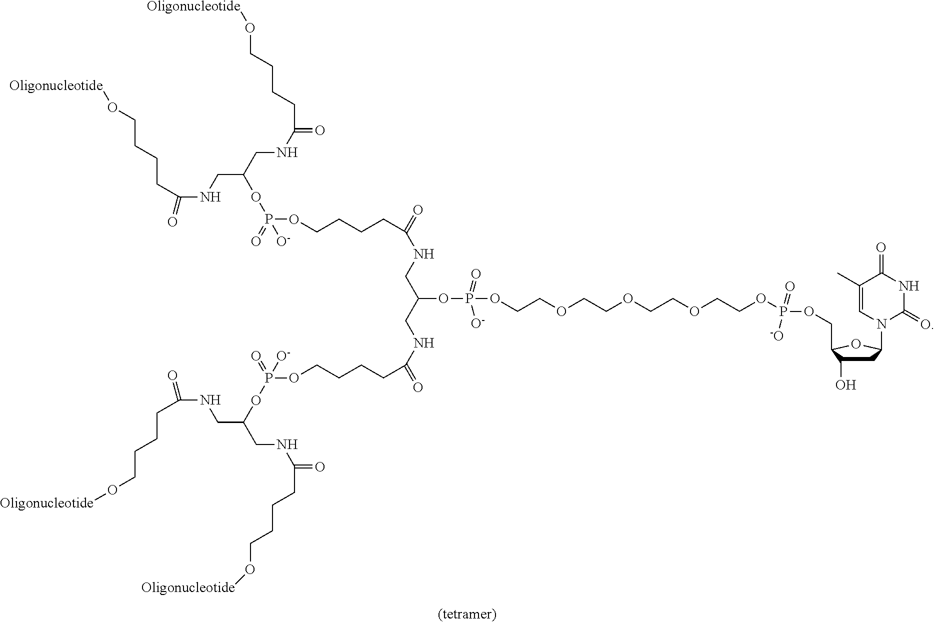

In certain embodiments, the antisense strand comprises about 15 nucleotides to about 25 nucleotides in length.

In certain embodiments, the sense strand comprises about 15 nucleotides to about 25 nucleotides in length.

In certain embodiments, the antisense strand is 20 nucleotides in length, 21 nucleotides in length, or 22 nucleotides in length.

In certain embodiments, the sense strand is 15 nucleotides in length, 16 nucleotides in length, 18 nucleotides in length, or 20 nucleotides in length.

In certain embodiments, the siRNA comprises a double-stranded region of about 15 base pairs to about 20 base pairs.

In certain embodiments, the siRNA comprises a double-stranded region of 15 base pairs, 16 base pairs, 18 base pairs, or 20 base pairs.

In certain embodiments, the siRNA comprises at least one blunt-end.

In certain embodiments, the siRNA comprises at least one single stranded nucleotide overhang.

In certain embodiments, the siRNA comprises naturally occurring nucleotides.

In certain embodiments, the siRNA comprises at least one modified nucleotide.

In certain embodiments, the modified nucleotide comprises a 2′-O-methyl modified nucleotide, a 2′-deoxy-2′-fluoro modified nucleotide, a 2′-deoxy-modified nucleotide, a locked nucleotide, an abasic nucleotide, a 2′-amino-modified nucleotide, a 2′-alkyl-modified nucleotide, a morpholino nucleotide, a phosphoramidate, a non-natural base comprising nucleotide, or a mixture thereof.

In certain embodiments, the siRNA comprises at least one modified internucleotide linkage.

In certain embodiments, the modified internucleotide linkage comprises phosphorothioate internucleotide linkage.

In certain embodiments, the siRNA comprises 4-16 phosphorothioate internucleotide linkages.

In certain embodiments, the siRNA comprises 8-13 phosphorothioate internucleotide linkages.

In certain embodiments, the siRNA comprises at least 80% chemically modified nucleotides.

In certain embodiments, the siRNA is fully chemically modified.

In certain embodiments, the sense strand comprises one or more nucleotide mismatches between the antisense strand and the sense strand.

In certain embodiments, the antisense strand comprises a 5′ phosphate, a 5′-alkyl phosphonate, a 5′ alkylene phosphonate, or a 5′ alkenyl phosphonate.

In certain embodiments, the antisense strand comprises a 5′ vinyl phosphonate.

In certain embodiments, the nucleotides at positions 1 and 2 from the 3′ end of sense strand, and the nucleotides at positions 1 and 2 from the 5′ end of antisense strand are connected to adjacent ribonucleotides via phosphorothioate linkages.

In one aspect, the disclosure provides a topical formulation comprising: (a) a plurality of microneedle particles; and (b) an oligonucleotide conjugate or branched oligonucleotide, wherein the oligonucleotide conjugate comprises: i) an oligonucleotide comprising a 5′ and a 3′ end and complementarity to a target nucleic acid; and ii) a functional moiety linked to the oligonucleotide (c) optionally a pharmaceutically acceptable carrier; and (d) optionally a pharmaceutically acceptable salt.

These and other aspects of the applicant's teachings are set forth herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Aspects, features, benefits, and advantages of the embodiments described herein will be apparent with regard to the following description, examples, claims, and accompanying drawings where:

FIGS. 1A-1B show schematic representations of oligonucleotide conjugates. FIG. 1A shows schematic representations of a dendron attached to a siRNA. FIG. 1B shows schematic representations of dendritic (D)-siRNAs comprising dendrons with hydrophilic (OH) and hydrophobic (CH3) end groups.

FIGS. 2A-2C show the high-performance liquid chromatography (HPLC) and size exclusion chromatography (SEC) analysis D-siRNA, DCA-siRNA, and siRNA. FIG. 2A shows the structure of the dendritic, docosanoic, and unconjugated siRNA tested for their hydrophobicity/retention time on HPLC, showing different retention times. FIG. 2B shows the reverse phase HPLC trace following the injection of D-siRNA or DCA-siRNA, showcasing the difference in hydrophobicity between them. FIG. 2C shows the results of an assay where D-siRNA, DCA-siRNA, and siRNA were injected in vivo via subcutaneous or intravenous injection in mice (n=2), and plasma was collected at 1 hour or 15 mins post injection. Plasma was run on size exclusion chromatography (SEC), Plasma proteins were monitored at 280 nm and Cy3 labeled oligonucleotides were monitored at 570 nm as previously reported.

FIG. 3 shows immunohistochemical images of microneedle particle treated human pannus skin samples. Oligonucleotide conjugates with DCA, dendrimer, EPA, and cholesterol, and a di-branched oligonucleotide (“Diol”) were delivered to the treated skin samples and detected through Cy3 labeling.

FIGS. 4A-4C show relative JAKI mRNA expression levels and siRNA accumulation in cells from microneedle particle treated skin samples followed by oligonucleotide conjugate delivery. DCA and dendrimer (“DD”) oligonucleotide conjugates were tested with and without microneedle particle treatment, and expression was measured in the epidermis (FIG. 4A) and dermis (FIG. 4B). siRNA accumulation was measured in immune cells, fibroblasts, endothelial cells, keratinocytes, and melanocytes. The assay was performed with a 1-minute STAR Particle treatment with about 40 kPa of pressure. 200 μL of 200 μM siRNA of each oligonucleotide was used, with a 96-hour incubation and 8 mm punch.

FIGS. 5A-5B show relative JAKI mRNA expression levels from skin samples treated with siRNA formulated with STAR particles. FIG. 5A shows the schematic of the application of the STAR particles and siRNA to the skin tissue and of the sampling, treatment, and analysis of the tissue. DCA and dendrimer (“D”) oligonucleotide conjugates were tested and expression was measured in the epidermis and dermis (FIG. 5B). About 3.5 mg (270 nmols) of siRNA formulated with star particles and excipient for a total of 225 mg was rubbed for about 60 seconds on a 3×3 cm zone and the analysis was performed after 4 days of application.

### DETAILED DESCRIPTION

It will be appreciated that for clarity, the following discussion will describe various aspects of embodiments of the applicant's teachings. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s).

The present disclosure relates to dendritic conjugates for the delivery of therapeutic oligonucleotides to the skin. In particular, the present disclosure provides compositions, systems, and methods for the delivery of therapeutic oligonucleotide conjugated to a dendron. The oligonucleotide conjugates disclosed herein can be delivered to the skin upon administration.

The disclosure also relates to dendritic conjugates for the delivery of therapeutic oligonucleotides to a tumor. In particular, the present disclosure provides compositions, systems, and methods for the delivery of therapeutic oligonucleotide conjugated to a dendron. The oligonucleotide conjugates disclosed herein can be delivered to the tumor upon administration.

The oligonucleotide conjugates described herein can promote simple, efficient, non-toxic delivery of oligonucleotides (e.g., siRNA, antisense oligonucleotide (ASO), macro-RNA), and promote potent silencing of therapeutic targets in skin cells and tumor in vivo.

Unless otherwise specified, nomenclature used in connection with cell and tissue culture, molecular biology, immunology, microbiology, genetics and protein and nucleic acid chemistry and hybridization described herein are those well-known and commonly used in the art. Unless otherwise specified, the methods and techniques provided herein are performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclature used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, delivery, and treatment of patients.

Unless otherwise defined herein, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting. So that the disclosure may be more readily understood, certain terms are first defined.

### Definitions

The use of the singular forms herein includes the plural unless specifically stated otherwise. As used herein, the singular forms “a,” “an,” and “the” include plural referents unless the context clearly dictates otherwise. Furthermore, use of the term “including” as well as other forms, such as “include,” “includes,” and “included,” is not limiting.

It is understood that wherever aspects are described herein with the language “comprising,” otherwise analogous aspects described in terms of “consisting of” and/or “consisting essentially of” are also provided.

As described herein, any concentration range, percentage range, ratio range or integer range is to be understood to include the value of any integer within the recited range and, when appropriate, fractions thereof (such as one tenth and one hundredth of an integer), unless otherwise indicated.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. When particular values or compositions are provided in the application and claims, unless otherwise stated, the meaning of “about” or “comprising essentially of” should be assumed to be within an acceptable error range for that particular value or composition.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As used herein in the context of oligonucleotide sequences, “A” represents a nucleoside comprising the base adenine (e.g., adenosine or a chemically-modified derivative thereof), “G” represents a nucleoside comprising the base guanine (e.g., guanosine or a chemically-modified derivative thereof), “U” represents a nucleoside comprising the base uracil (e.g., uridine or a chemically-modified derivative thereof), and “C” represents a nucleoside comprising the base adenine (e.g., cytidine or a chemically-modified derivative thereof).

The term “nucleoside” refers to a molecule having a purine or pyrimidine base covalently linked to a ribose or deoxyribose sugar. Exemplary nucleosides include adenosine, guanosine, cytidine, uridine and thymidine. Additional exemplary nucleosides include inosine, 1-methyl inosine, pseudouridine, 5,6-dihydrouridine, ribothymidine, 2N-methylguanosine and N2,N2-dimethylguanosine (also referred to as “rare” nucleosides). The term “nucleotide” refers to a nucleoside having one or more phosphate groups joined in ester linkages to the sugar moicty. Exemplary nucleotides include nucleoside monophosphates, diphosphates and triphosphates. The terms “polynucleotide” and “nucleic acid molecule” are used interchangeably herein and refer to a polymer of nucleotides joined together by a phosphodiester or phosphorothioate linkage between 5′ and 3′ carbon atoms.

The term “RNA” or “RNA molecule” or “ribonucleic acid molecule” refers to a polymer of ribonucleotides (e.g., 2, 3, 4, 5, 10, 15, 20, 25, 30, or more ribonucleotides). The term “DNA” or “DNA molecule” or “deoxyribonucleic acid molecule” refers to a polymer of deoxyribonucleotides. DNA and RNA can be synthesized naturally (e.g., by DNA replication or transcription of DNA, respectively). RNA can be post-transcriptionally modified. DNA and RNA can also be chemically synthesized. DNA and RNA can be single-stranded (i.e., ssRNA and ssDNA, respectively) or multi-stranded (e.g., double stranded, i.e., dsRNA and dsDNA, respectively). “mRNA” or “messenger RNA” is single-stranded RNA that specifies the amino acid sequence of one or more polypeptide chains. This information is translated during protein synthesis when ribosomes bind to the mRNA.

As used herein, the term “small interfering RNA” (“siRNA”) (also referred to in the art as “short interfering RNAs”) refers to an RNA (or RNA analog) comprising between about 10-50 nucleotides (or nucleotide analogs), which is capable of directing or mediating RNA interference. The siRNA is a duplex formed by a sense strand and antisense strand which have sufficient complementarity to each other to form said duplex. In certain embodiments, a siRNA comprises between about 15-30 nucleotides or nucleotide analogs, or between about 16-25 nucleotides (or nucleotide analogs), or between about 18-23 nucleotides (or nucleotide analogs), or between about 19-22 nucleotides (or nucleotide analogs) (e.g., 19, 20, 21 or 22 nucleotides or nucleotide analogs). The term “short” siRNA refers to a siRNA comprising about 21 nucleotides (or nucleotide analogs), for example, 19, 20, 21 or 22 nucleotides. The term “long” siRNA refers to a siRNA comprising about 24-25 nucleotides, for example, 23, 24, 25 or 26 nucleotides. Short siRNAs may, in some instances, include fewer than 19 nucleotides, e.g., 16, 17 or 18 nucleotides, provided that the shorter siRNA retains the ability to mediate RNAi. Likewise, long siRNAs may, in some instances, include more than 26 nucleotides, provided that the longer siRNA retains the ability to mediate RNAi absent further processing, e.g., enzymatic processing, to a short siRNA.

The term “nucleotide analog” or “altered nucleotide” or “modified nucleotide” or “chemically modified nucleotide” refers to a non-standard nucleotide, including non-naturally occurring ribonucleotides or deoxyribonucleotides. Exemplary nucleotide analogs are modified at any position so as to alter certain chemical properties of the nucleotide yet retain the ability of the nucleotide analog to perform its intended function. Examples of positions of the nucleotide, which may be derivatized include: the 5 position, e.g., 5-(2-amino) propyl uridine, 5-bromo uridine, 5-propyne uridine, 5-propenyl uridine, etc.; the 6 position, e.g., 6-(2-amino) propyl uridine; and the 8-position for adenosine and/or guanosines, e.g., 8-bromo guanosine, 8-chloro guanosine, 8-fluoroguanosine, etc. Nucleotide analogs also include deaza nucleotides, e.g., 7-deaza-adenosine; O-and N-modified (e.g., alkylated, e.g., N6-methyl adenosine, or as otherwise known in the art) nucleotides; and other heterocyclically modified nucleotide analogs, such as those described in Herdewijn, Antisense Nucleic Acid Drug Dev., 2000 Aug. 10 (4): 297-310.

Nucleotide analogs may also comprise modifications to the sugar portion of the nucleotides. For example, the 2′ OH-group may be replaced by a group selected from H, OR, R, F, Cl, Br, I, SH, SR, NH₂, NHR, NR₂, or COOR, wherein R is substituted or unsubstituted C₁-C₆ alkyl, alkenyl, alkynyl, aryl, etc. Other possible modifications include those described in U.S. Pat. Nos. 5,858,988, and 6,291,438. In certain embodiments, the nucleotide analog comprises a 2′-O-methyl modification. In certain embodiments, the nucleotide analog comprises a 2′-fluoro modification.

The phosphate group of the nucleotide may also be modified, e.g., by substituting one or more of the oxygens of the phosphate group with sulfur (e.g., phosphorothioate), or by making other substitutions, which allow the nucleotide to perform its intended function, such as described in, for example, Eckstein, Antisense Nucleic Acid Drug Dev. 2000 Apr. 10 (2): 117-21, Rusckowski et al. Antisense Nucleic Acid Drug Dev. 2000 Oct. 10 (5): 333-45, Stein, Antisense Nucleic Acid Drug Dev. 2001 Oct. 11 (5): 317-25, Vorobjev et al. Antisense Nucleic Acid Drug Dev. 2001 Apr. 11 (2): 77-85, and U.S. Pat. No. 5,684,143. Certain of the above-referenced modifications (e.g., phosphate group modifications) decrease the rate of hydrolysis of, for example, polynucleotides comprising said analogs in vivo or in vitro.

The term “RNA analog” refers to a polynucleotide (e.g., a chemically synthesized polynucleotide) having at least one altered or modified nucleotide as compared to a corresponding unaltered or unmodified RNA, but retaining the same or similar nature or function as the corresponding unaltered or unmodified RNA. As discussed above, the oligonucleotides may be linked with linkages, which result in a lower rate of hydrolysis of the RNA analog as compared to an RNA molecule with phosphodiester linkages. For example, the nucleotides of the analog may comprise methylenediol, ethylene diol, oxymethylthio, oxyethylthio, oxycarbonyloxy, phosphorodiamidate, phosphoroamidate, and/or phosphorothioate linkages. Some RNA analogues include sugar-and/or backbone-modified ribonucleotides and/or deoxyribonucleotides. Such alterations or modifications can further include addition of non-nucleotide material, such as to the end(s) of the RNA or internally (at one or more nucleotides of the RNA). An RNA analog need only be sufficiently similar to natural RNA that it has the ability to mediate RNA interference.

As used herein, the term “RNA interference” (“RNAi”) refers to a selective intracellular degradation of RNA. RNAi occurs in cells naturally to remove foreign RNAs (e.g., viral RNAs). Natural RNAi proceeds via fragments cleaved from free dsRNA, which direct the degradative mechanism to other similar RNA sequences. Alternatively, RNAi can be initiated by the hand of man, for example, to silence the expression of target genes.

An RNAi agent, e.g., an RNA silencing agent, having a strand, which is “sequence sufficiently complementary to a target mRNA sequence to direct target-specific RNA interference (RNAi)” means that the strand has a sequence sufficient to trigger the destruction of the target mRNA by the RNAi machinery or process.

As used herein, the term “isolated RNA” (e.g., “isolated siRNA” or “isolated siRNA precursor”) refers to RNA molecules, which are substantially free of other cellular material, or culture medium when produced by recombinant techniques, or substantially free of chemical precursors or other chemicals when chemically synthesized.

As used herein, the term “RNA silencing” refers to a group of sequence-specific regulatory mechanisms (e.g., RNA interference (RNAi), transcriptional gene silencing (TGS), post-transcriptional gene silencing (PTGS), quelling, co-suppression, and translational repression) mediated by RNA molecules, which result in the inhibition or “silencing” of the expression of a corresponding protein-coding gene. RNA silencing has been observed in many types of organisms, including plants, animals, and fungi.

The term “in vitro” has its art recognized meaning, e.g., involving purified reagents or extracts, e.g., cell extracts. The term “in vivo” also has its art recognized meaning, e.g., involving living cells, e.g., immortalized cells, primary cells, cell lines, and/or cells in an organism.

As used herein, a “target” refers to a particular nucleic acid sequence (e.g., a gene, an mRNA, a miRNA or the like) that an oligonucleotide conjugate or branched oligonucleotide of the disclosure binds to and/or otherwise effects the expression of. In certain embodiments, the target is expressed in the eye. In certain embodiments, target is expressed in a specific eye cell. In other embodiments, a target is associated with a particular disease or disorder in a subject.

As used herein, the term “target gene” is a gene whose expression is to be substantially inhibited or “silenced.” This silencing can be achieved by RNA silencing, e.g., by cleaving the mRNA of the target gene or translational repression of the target gene. The term “non-target gene” is a gene whose expression is not to be substantially silenced. In one embodiment, the polynucleotide sequences of the target and non-target gene (e.g., mRNA encoded by the target and non-target genes) can differ by one or more nucleotides. In another embodiment, the target and non-target genes can differ by one or more polymorphisms (e.g., Single Nucleotide Polymorphisms or SNPs). In another embodiment, the target and non-target genes can share less than 100% sequence identity. In another embodiment, the non-target gene may be a homologue (e.g., an orthologue or paralogue) of the target gene.

As used herein, the term “RNA silencing agent” refers to an RNA, which is capable of inhibiting or “silencing” the expression of a target gene. In certain embodiments, the RNA silencing agent is capable of preventing complete processing (e.g., the full translation and/or expression) of a mRNA molecule through a post-transcriptional silencing mechanism. RNA silencing agents include small (<50 b.p.), noncoding RNA molecules, for example RNA duplexes comprising paired strands, as well as precursor RNAs from which such small non-coding RNAs can be generated. Exemplary RNA silencing agents include siRNAs, miRNAs, siRNA-like duplexes, antisense oligonucleotides, GAPMER molecules, and dual-function oligonucleotides, as well as precursors thereof. In one embodiment, the RNA silencing agent is capable of inducing RNA interference. In another embodiment, the RNA silencing agent is capable of mediating translational repression.

As used herein, the term “rare nucleotide” refers to a naturally occurring nucleotide that occurs infrequently, including naturally occurring deoxyribonucleotides or ribonucleotides that occur infrequently, e.g., a naturally occurring ribonucleotide that is not guanosine, adenosine, cytosine, or uridine. Examples of rare nucleotides include, but are not limited to, inosine, 1-methyl inosine, pseudouridine, 5,6-dihydrouridine, ribothymidine, 2N-methylguanosine and 2,2N,N-dimethylguanosine.

The term “engineered,” as in an engineered RNA precursor, or an engineered nucleic acid molecule, indicates that the precursor or molecule is not found in nature, in that all or a portion of the nucleic acid sequence of the precursor or molecule is created or selected by a human. Once created or selected, the sequence can be replicated, translated, transcribed, or otherwise processed by mechanisms within a cell. Thus, an RNA precursor produced within a cell from a transgene that includes an engineered nucleic acid molecule is an engineered RNA precursor.

As used herein, the term “microRNA” (“miRNA”), also known in the art as “small temporal RNAs” (“stRNAs”), refers to a small (10-50 nucleotide) RNA, which are genetically encoded (e.g., by viral, mammalian, or plant genomes) and are capable of directing or mediating RNA silencing. An “miRNA disorder” shall refer to a disease or disorder characterized by an aberrant expression or activity of a miRNA.

As used herein, the term “dual functional oligonucleotide” refers to an RNA silencing agent having the formula T-L-μ, wherein T is an mRNA targeting moiety, L is a linking moiety, and μ is a miRNA recruiting moiety. As used herein, the terms “mRNA targeting moiety,” “targeting moiety,” “mRNA targeting portion” or “targeting portion” refer to a domain, portion or region of the dual functional oligonucleotide having sufficient size and sufficient complementarity to a portion or region of an mRNA chosen or targeted for silencing (i.e., the moiety has a sequence sufficient to capture the target mRNA).

As used herein, the term “linking moiety” or “linking portion” refers to a domain, portion or region of the RNA-silencing agent which covalently joins or links the mRNA.

As used herein, the term “antisense strand” of an RNA silencing agent, e.g., an siRNA, refers to a strand that is substantially complementary to a section of about 10-50 nucleotides, e.g., about 15-30, 16-25, 18-23 or 19-22 nucleotides of the mRNA of the gene targeted for silencing. The antisense strand or first strand has sequence sufficiently complementary to the desired target mRNA sequence to direct target-specific silencing, e.g., complementarity sufficient to trigger the destruction of the desired target mRNA by the RNAi machinery or process (RNAi interference) or complementarity sufficient to trigger translational repression of the desired target mRNA.

The term “sense strand” or “second strand” of an RNA silencing agent, e.g., an siRNA or RNA silencing agent, refers to a strand that is complementary to the antisense strand or first strand. Antisense and sense strands can also be referred to as first or second strands, the first or second strand having complementarity to the target sequence and the respective second or first strand having complementarity to said first or second strand. miRNA duplex intermediates or siRNA-like duplexes include a miRNA strand having sufficient complementarity to a section of about 10-50 nucleotides of the mRNA of the gene targeted for silencing and a miRNA* strand having sufficient complementarity to form a duplex with the miRNA strand.

As used herein, the term “guide strand” refers to a strand of an RNA silencing agent, e.g., an antisense strand of an siRNA duplex or siRNA sequence, that enters into the RISC complex and directs cleavage of the target mRNA.

As used herein, the term “asymmetry,” as in the asymmetry of the duplex region of an RNA silencing agent (e.g., the stem of an shRNA), refers to an inequality of bond strength or base pairing strength between the termini of the RNA silencing agent (e.g., between terminal nucleotides on a first strand or stem portion and terminal nucleotides on an opposing second strand or stem portion), such that the 5′ end of one strand of the duplex is more frequently in a transient unpaired, e.g., single-stranded, state than the 5′ end of the complementary strand. This structural difference determines that one strand of the duplex is preferentially incorporated into a RISC complex. The strand whose 5′ end is less tightly paired to the complementary strand will preferentially be incorporated into RISC and mediate RNAi.

As used herein, the term “bond strength” or “base pair strength” refers to the strength of the interaction between pairs of nucleotides (or nucleotide analogs) on opposing strands of an oligonucleotide duplex (e.g., an siRNA duplex), due primarily to H-bonding, van der Waals interactions, and the like, between said nucleotides (or nucleotide analogs).

As used herein, the “5′ end,” as in the 5′ end of an antisense strand, refers to the 5′ terminal nucleotides, e.g., between one and about 5 nucleotides at the 5′ terminus of the antisense strand. As used herein, the “3′ end,” as in the 3′ end of a sense strand, refers to the region, e.g., a region of between one and about 5 nucleotides, that is complementary to the nucleotides of the 5′ end of the complementary antisense strand.

As used herein the term “destabilizing nucleotide” refers to a first nucleotide or nucleotide analog capable of forming a base pair with second nucleotide or nucleotide analog such that the base pair is of lower bond strength than a conventional base pair (i.e., Watson-Crick base pair). In certain embodiments, the destabilizing nucleotide is capable of forming a mismatch base pair with the second nucleotide. In other embodiments, the destabilizing nucleotide is capable of forming a wobble base pair with the second nucleotide. In yet other embodiments, the destabilizing nucleotide is capable of forming an ambiguous base pair with the second nucleotide.

As used herein, the term “base pair” refers to the interaction between pairs of nucleotides (or nucleotide analogs) on opposing strands of an oligonucleotide duplex (e.g., a duplex formed by a strand of an RNA silencing agent and a target mRNA sequence), due primarily to H-bonding, van der Waals interactions, and the like between said nucleotides (or nucleotide analogs). As used herein, the term “bond strength” or “base pair strength” refers to the strength of the base pair.

As used herein, the term “mismatched base pair” refers to a base pair consisting of non-complementary or non-Watson-Crick base pairs, for example, not normal complementary G:C, A:T or A:U base pairs. As used herein the term “ambiguous base pair” (also known as a non-discriminatory base pair) refers to a base pair formed by a universal nucleotide.

As used herein, term “universal nucleotide” (also known as a “neutral nucleotide”) include those nucleotides (e.g., certain destabilizing nucleotides) having a base (a “universal base” or “neutral base”) that does not significantly discriminate between bases on a complementary polynucleotide when forming a base pair. Universal nucleotides are predominantly hydrophobic molecules that can pack efficiently into antiparallel duplex nucleic acids (e.g., double-stranded DNA or RNA) due to stacking interactions. The base portion of universal nucleotides typically comprise a nitrogen-containing aromatic heterocyclic moicty.

As used herein, the terms “sufficient complementarity” or “sufficient degree of complementarity” mean that the RNA silencing agent has a sequence (e.g., in the antisense strand, mRNA targeting moiety or miRNA recruiting moiety), which is sufficient to bind the desired target RNA, respectively, and to trigger the RNA silencing of the target mRNA.

As used herein, the term “translational repression” refers to a selective inhibition of mRNA translation. Natural translational repression proceeds via miRNAs cleaved from shRNA precursors. Both RNAi and translational repression are mediated by RISC. Both RNAi and translational repression occur naturally or can be initiated by the hand of man, for example, to silence the expression of target genes.

Various methodologies of the instant disclosure include a step that involves comparing a value, level, feature, characteristic, property, etc. to a “suitable control,” referred to interchangeably herein as an “appropriate control.” A “suitable control” or “appropriate control” is any control or standard familiar to one of ordinary skill in the art useful for comparison purposes. In one embodiment, a “suitable control” or “appropriate control” is a value, level, feature, characteristic, property, etc. determined prior to performing an RNAi methodology, as described herein. For example, a transcription rate, mRNA level, translation rate, protein level, biological activity, cellular characteristic or property, genotype, phenotype, etc. can be determined prior to introducing an RNA silencing agent of the disclosure into a cell or organism. In another embodiment, a “suitable control” or “appropriate control” is a value, level, feature, characteristic, property, etc. determined in a cell or organism, e.g., a control or normal cell or organism, exhibiting, for example, normal traits. In yet another embodiment, a “suitable control” or “appropriate control” is a predefined value, level, feature, characteristic, property, etc.

### Design of siRNA Molecules

The oligonucleotide conjugates described herein comprise an oligonucleotide. Non-limiting examples of oligonucleotide include siRNA, antisense oligonucleotide (ASO), and macro-RNA.

In some embodiments, an siRNA molecule of the invention is a duplex consisting of a sense strand and complementary antisense strand, the antisense strand having sufficient complementary to a target sequence such as a mRNA sequence (e.g., a htt mRNA sequence, cyclophilin B mRNA sequence, etc.) to mediate RNAi. Preferably, the siRNA molecule has a length from about 10-50 or more nucleotides, i.e., cach strand comprises 10-50 nucleotides (or nucleotide analogs). More preferably, the siRNA molecule has a length from about 16-30, e.g., 16, 17, 18, 19, 20, 21, 22, 23, 24, 25, 26, 27, 28, 29, or 30 nucleotides in cach strand, wherein one of the strands is sufficiently complementary to a target region. Preferably, the strands are aligned such that there are at least 1, 2, or 3 bases at the end of the strands which do not align (i.e., for which no complementary bases occur in the opposing strand) such that an overhang of 1, 2 or 3 residues occurs at one or both ends of the duplex when strands are annealed. Preferably, the siRNA molecule has a length from about 10-50 or more nucleotides, i.e., each strand comprises 10-50 nucleotides (or nucleotide analogs). More preferably, the siRNA molecule has a length from about 16-30, e.g., 16, 17, 18, 19, 20, 21, 22, 23, 24, 25, 26, 27, 28, 29, or 30 nucleotides in cach strand, wherein one of the strands is substantially complementary to a target sequence, and the other strand is identical or substantially identical to the first strand.

Generally, siRNAs can be designed by using any method known in the art, for instance, by using the following protocol:
1. The siRNA may be specific for a target sequence. Preferably, the first strand is substantially complementary to the target sequence, and the other strand is substantially complementary to the first strand. In an embodiment, the target sequence is outside a coding region of the target gene. Exemplary target sequences are selected from the 5′ untranslated region (5′-UTR) or an intronic region of a target gene. Cleavage of mRNA at these sites should eliminate translation of corresponding mutant protein. Target sequences from other regions of the htt gene are also suitable for targeting. A sense strand is designed based on the target sequence. Further, siRNAs with lower G/C content (35-55%) may be more active than those with G/C content higher than 55%. Thus, in one embodiment, the invention includes nucleic acid molecules having 35-55% G/C content.
2. The sense strand of the siRNA is designed based on the sequence of the selected target site. Preferably the RNA silencing agents of the invention do not elicit a PKR response (i.e., are of a sufficiently short length). However, longer RNA silencing agents may be useful, for example, in cell types incapable of generating a PRK response or in situations where the PKR response has been down-regulated or dampened by alternative means.

The siRNA molecules of the invention have sufficient complementarity with the target sequence such that the siRNA can mediate RNAi. In general, siRNA containing nucleotide sequences sufficiently identical to a target sequence portion of the target gene to effect RISC-mediated cleavage of the target gene are preferred. Accordingly, in a preferred embodiment, the sense strand of the siRNA is designed have to have a sequence sufficiently identical to a portion of the target. For example, the sense strand may have 100% identity to the target site. However, 100% identity is not required. Greater than 80% identity, e.g., 80%, 81%, 82%, 83%, 84%, 85%, 86%, 87%, 88%, 89%, 90%, 91%, 92%, 93%, 94%, 95%, 96%, 97%, 98%, 99% or even 100% identity, between the sense strand and the target RNA sequence is preferred. The invention has the advantage of being able to tolerate certain sequence variations to enhance efficiency and specificity of RNAi. In one embodiment, the sense strand has 4, 3, 2, 1, or 0 mismatched nucleotide(s) with a target region, such as a target region that differs by at least one base pair between a wild-type and mutant allele, e.g., a target region comprising the gain-of-function mutation, and the other strand is identical or substantially identical to the first strand. Moreover, siRNA sequences with small insertions or deletions of 1 or 2 nucleotides may also be effective for mediating RNAi. Alternatively, siRNA sequences with nucleotide analog substitutions or insertions can be effective for inhibition.

Sequence identity may be determined by sequence comparison and alignment algorithms known in the art. To determine the percent identity of two nucleic acid sequences (or of two amino acid sequences), the sequences are aligned for optimal comparison purposes (e.g., gaps can be introduced in the first sequence or second sequence for optimal alignment). The nucleotides (or amino acid residues) at corresponding nucleotide (or amino acid) positions are then compared. When a position in the first sequence is occupied by the same residue as the corresponding position in the second sequence, then the molecules are identical at that position. The percent identity between the two sequences is a function of the number of identical positions shared by the sequences (i.e., percent (%) homology=number of identical positions/total number of positions×100), optionally penalizing the score for the number of gaps introduced and/or length of gaps introduced.

The comparison of sequences and determination of percent identity between two sequences can be accomplished using a mathematical algorithm. In one embodiment, the alignment generated over a certain portion of the sequence aligned having sufficient identity but not over portions having low degree of identity (i.e., a local alignment). A preferred, non-limiting example of a local alignment algorithm utilized for the comparison of sequences is the algorithm of Karlin and Altschul (1990) Proc. Natl. Acad. Sci. USA 87:2264-68, modified as in Karlin and Altschul (1993) Proc. Natl. Acad. Sci. USA 90:5873-77. Such an algorithm is incorporated into the BLAST programs (version 2.0) of Altschul, et al. (1990) J. Mol. Biol. 215:403-10.

In another embodiment, the alignment is optimized by introducing appropriate gaps and percent identity is determined over the length of the aligned sequences (i.e., a gapped alignment). To obtain gapped alignments for comparison purposes, Gapped BLAST can be utilized as described in Altschul et al., (1997) Nucleic Acids Res. 25 (17): 3389-3402. In another embodiment, the alignment is optimized by introducing appropriate gaps and percent identity is determined over the entire length of the sequences aligned (i.e., a global alignment). A preferred, non-limiting example of a mathematical algorithm utilized for the global comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.
3. The antisense or guide strand of the siRNA is routinely the same length as the sense strand and includes complementary nucleotides. In one embodiment, the guide and sense strands are fully complementary, i.e., the strands are blunt ended when aligned or annealed. In another embodiment, the strands of the siRNA can be paired in such a way as to have a 3′ overhang of 1 to 4, e.g., 2, nucleotides. Overhangs can comprise (or consist of) nucleotides corresponding to the target gene sequence (or complement thereof). Alternatively, overhangs can comprise (or consist of) deoxyribonucleotides, for example dTs, or nucleotide analogs, or other suitable non-nucleotide material. Thus, in another embodiment, the nucleic acid molecules may have a 3′ overhang of 2 nucleotides, such as TT. The overhanging nucleotides may be either RNA or DNA. As noted above, it is desirable to choose a target region wherein the mutant: wild type mismatch is a purine: purine mismatch.
4. Using any method known in the art, compare the potential targets to the appropriate genome database (human, mouse, rat, etc.) and eliminate from consideration any target sequences with significant homology to other coding sequences. One such method for such sequence homology searches is known as BLAST, which is available at National Center for Biotechnology Information website.
5. Select one or more sequences that meet the criteria for evaluation.

Further general information about the design and use of siRNA may be found in “The siRNA User Guide,” available at The Max-Plank-Institut fur Biophysikalishe Chemie website.

Alternatively, the siRNA may be defined functionally as a nucleotide sequence (or oligonucleotide sequence) that is capable of hybridizing with the target sequence (e.g., 400 mM NaCl, 40 mM PIPES pH 6.4, 1 mM EDTA, 50° C. or 70° C. hybridization for 12-16 hours; followed by washing). Additional preferred hybridization conditions include hybridization at 70° C. in 1xSSC or 50° C. in 1xSSC, 50% formamide followed by washing at 70° C. in 0.3xSSC or hybridization at 70° C. in 4xSSC or 50° C. in 4xSSC, 50% formamide followed by washing at 67° C. in 1xSSC. The hybridization temperature for hybrids anticipated to be less than 50 base pairs in length should be 5-10° C. less than the melting temperature (Tm) of the hybrid, where Tm is determined according to the following equations. For hybrids less than 18 base pairs in length, Tm(°C)=2 (#of A+T bases)+4 (#of G+C bases). For hybrids between 18 and 49 base pairs in length, Tm (°C)=81.5+16.6 (log 10 [Na+])+0.41 (% G+C)−(600/N), where N is the number of bases in the hybrid, and [Na+] is the concentration of sodium ions in the hybridization buffer ([Na+] for 1xSSC=0.165 M). Additional examples of stringency conditions for polynucleotide hybridization are provided in Sambrook, J., E. F. Fritsch, and T. Maniatis, 1989, Molecular Cloning: A Laboratory Manual, Cold Spring Harbor Laboratory Press, Cold Spring Harbor, N.Y., chapters 9 and 11, and Current Protocols in Molecular Biology, 1995, F. M. Ausubel et al., eds., John Wiley & Sons, Inc., sections 2.10 and 6.3-6.4, incorporated herein by reference.

Negative control siRNAs should have the same nucleotide composition as the selected siRNA, but without significant sequence complementarity to the appropriate genome. Such negative controls may be designed by randomly scrambling the nucleotide sequence of the selected siRNA. A homology search can be performed to ensure that the negative control lacks homology to any other gene in the appropriate genome. In addition, negative control siRNAs can be designed by introducing one or more base mismatches into the sequence.
6. To validate the effectiveness by which siRNAs destroy target mRNAs (e.g., wild-type or mutant huntingtin mRNA), the siRNA may be incubated with target cDNA (e.g., huntingtin cDNA) in a Drosophila-based in vitro mRNA expression system. Radiolabeled with ³²P, newly synthesized target mRNAs (e.g., huntingtin mRNA) are detected autoradiographically on an agarose gel. The presence of cleaved target mRNA indicates mRNA nuclease activity. Suitable controls include omission of siRNA and use of non-target cDNA. Alternatively, control siRNAs are selected having the same nucleotide composition as the selected siRNA, but without significant sequence complementarity to the appropriate target gene. Such negative controls can be designed by randomly scrambling the nucleotide sequence of the selected siRNA. A homology search can be performed to ensure that the negative control lacks homology to any other gene in the appropriate genome. In addition, negative control siRNAs can be designed by introducing one or more base mismatches into the sequence.

siRNAs may be designed to target any of the target sequences described supra. Said siRNAs comprise an antisense strand which is sufficiently complementary with the target sequence to mediate silencing of the target sequence. In certain embodiments, the RNA silencing agent is a siRNA.

Sites of siRNA-mRNA complementation are selected which result in optimal mRNA specificity and maximal mRNA cleavage.

### siRNA-Like Molecules

siRNA-like molecules of the invention have a sequence (i.e., have a strand having a sequence) that is “sufficiently complementary” to a target sequence of an mRNA (e.g., htt mRNA) to direct gene silencing either by RNAi or translational repression. siRNA-like molecules are designed in the same way as siRNA molecules, but the degree of sequence identity between the sense strand and target RNA approximates that observed between an miRNA and its target. In general, as the degree of sequence identity between a miRNA sequence and the corresponding target gene sequence is decreased, the tendency to mediate post-transcriptional gene silencing by translational repression rather than RNAi is increased. Therefore, in an alternative embodiment, where post-transcriptional gene silencing by translational repression of the target gene is desired, the miRNA sequence has partial complementarity with the target gene sequence. In certain embodiments, the miRNA sequence has partial complementarity with one or more short sequences (complementarity sites) dispersed within the target mRNA (e.g., within the 3′-UTR of the target mRNA) (Hutvagner and Zamore, Science, 2002; Zeng et al., Mol. Cell, 2002; Zeng et al., RNA, 2003; Doench et al., Genes & Dev., 2003). Since the mechanism of translational repression is cooperative, multiple complementarity sites (e.g., 2, 3, 4, 5, or 6) may be targeted in certain embodiments.

The capacity of a siRNA-like duplex to mediate RNAi or translational repression may be predicted by the distribution of non-identical nucleotides between the target gene sequence and the nucleotide sequence of the silencing agent at the site of complementarity. In one embodiment, where gene silencing by translational repression is desired, at least one non-identical nucleotide is present in the central portion of the complementarity site so that duplex formed by the miRNA guide strand and the target mRNA contains a central “bulge” (Doench J G et al., Genes & Dev., 2003). In another embodiment 2, 3, 4, 5, or 6 contiguous or non-contiguous non-identical nucleotides are introduced. The non-identical nucleotide may be selected such that it forms a wobble base pair (e.g., G: U) or a mismatched base pair (G:A, C:A, C:U, G:G, A:A, C:C, U:U). In a further preferred embodiment, the “bulge” is centered at nucleotide positions 12 and 13 from the 5′ end of the miRNA molecule.

### Modified RNA Silencing Agents

In certain aspects of the invention, an RNA silencing agent (or any portion thereof) of the invention as described supra may be modified such that the activity of the agent is further improved. For example, the RNA silencing agents described in above may be modified with any of the modifications described infra. The modifications can, in part, serve to further enhance target discrimination, to enhance stability of the agent (e.g., to prevent degradation), to promote cellular uptake, to enhance the target efficiency, to improve efficacy in binding (e.g., to the targets), to improve patient tolerance to the agent, and/or to reduce toxicity.

### 1) Modifications to Enhance Target Discrimination

In certain embodiments, the RNA silencing agents of the invention may be substituted with a destabilizing nucleotide to enhance single nucleotide target discrimination (see U.S. application Ser. No. 11/698,689, filed Jan. 25, 2007, and U.S. Provisional Application No. 60/762,225 filed Jan. 25, 2006, both of which are incorporated herein by reference). Such a modification may be sufficient to abolish the specificity of the RNA silencing agent for a non-target mRNA (e.g., wild-type mRNA), without appreciably affecting the specificity of the RNA silencing agent for a target mRNA (e.g., gain-of-function mutant mRNA).

In preferred embodiments, the RNA silencing agents of the invention are modified by the introduction of at least one universal nucleotide in the antisense strand thereof. Universal nucleotides comprise base portions that are capable of base pairing indiscriminately with any of the four conventional nucleotide bases (e.g., A, G, C, U). A universal nucleotide is preferred because it has relatively minor effect on the stability of the RNA duplex, or the duplex formed by the guide strand of the RNA silencing agent and the target mRNA. Exemplary universal nucleotide includes those having an inosine base portion or an inosine analog base portion selected from the group consisting of deoxyinosine (e.g., 2′-deoxyinosine), 7-deaza-2′-deoxyinosine, 2′-aza-2′-deoxyinosine, PNA-inosine, morpholino-inosine, LNA-inosine, phosphoramidate-inosine, 2′-O-methoxyethyl-inosine, and 2′-O-Me-inosine. In particularly preferred embodiments, the universal nucleotide is an inosine residue or a naturally occurring analog thereof.

In certain embodiments, the RNA silencing agents of the invention are modified by the introduction of at least one destabilizing nucleotide within 5 nucleotides from a specificity-determining nucleotide (i.e., the nucleotide which recognizes the disease-related polymorphism). For example, the destabilizing nucleotide may be introduced at a position that is within 5, 4, 3, 2, or 1 nucleotide(s) from a specificity-determining nucleotide. In exemplary embodiments, the destabilizing nucleotide is introduced at a position which is 3 nucleotides from the specificity-determining nucleotide (i.e., such that there are 2 stabilizing nucleotides between the destabilizing nucleotide and the specificity-determining nucleotide). In RNA silencing agents having two strands or strand portions (e.g., siRNAs and shRNAs), the destabilizing nucleotide may be introduced in the strand or strand portion that does not contain the specificity-determining nucleotide. In preferred embodiments, the destabilizing nucleotide is introduced in the same strand or strand portion that contains the specificity-determining nucleotide.

### 2) Modifications to Enhance Efficacy and Specificity

In certain embodiments, the RNA silencing agents of the invention may be altered to facilitate enhanced efficacy and specificity in mediating RNAi according to asymmetry design rules (see U.S. Pat. Nos. 8,309,704, 7,750,144, 8,304,530, 8,329,892 and 8,309,705). Such alterations facilitate entry of the antisense strand of the siRNA (e.g., a siRNA designed using the methods of the invention or an siRNA produced from a shRNA) into RISC in favor of the sense strand, such that the antisense strand preferentially guides cleavage or translational repression of a target mRNA, and thus increasing or improving the efficiency of target cleavage and silencing. Preferably the asymmetry of an RNA silencing agent is enhanced by lessening the base pair strength between the antisense strand 5′ end (AS 5′) and the sense strand 3′ end (S 3′) of the RNA silencing agent relative to the bond strength or base pair strength between the antisense strand 3′ end (AS 3′) and the sense strand 5′ end (S ′5) of said RNA silencing agent.

In one embodiment, the asymmetry of an RNA silencing agent of the invention may be enhanced such that there are fewer G: C base pairs between the 5′ end of the first or antisense strand and the 3′ end of the sense strand portion than between the 3′ end of the first or antisense strand and the 5′ end of the sense strand portion. In another embodiment, the asymmetry of an RNA silencing agent of the invention may be enhanced such that there is at least one mismatched base pair between the 5′ end of the first or antisense strand and the 3′ end of the sense strand portion. Preferably, the mismatched base pair is selected from the group consisting of G:A, C:A, C:U, G:G, A:A, C:C and U:U. In another embodiment, the asymmetry of an RNA silencing agent of the invention may be enhanced such that there is at least one wobble base pair, e.g., G:U, between the 5′ end of the first or antisense strand and the 3′ end of the sense strand portion. In another embodiment, the asymmetry of an RNA silencing agent of the invention may be enhanced such that there is at least one base pair comprising a rare nucleotide, e.g., inosine (I). Preferably, the base pair is selected from the group consisting of an I:A, I:U and I:C. In yet another embodiment, the asymmetry of an RNA silencing agent of the invention may be enhanced such that there is at least one base pair comprising a modified nucleotide. In preferred embodiments, the modified nucleotide is selected from the group consisting of 2-amino-G, 2-amino-A, 2,6-diamino-G, and 2,6-diamino-A.

3) RNA Silencing Agents with Enhanced Stability

The RNA silencing agents of the present invention can be modified to improve stability in serum or in growth medium for cell cultures. In order to enhance the stability, the 3′-residues may be stabilized against degradation, e.g., they may be selected such that they consist of purine nucleotides, particularly adenosine or guanosine nucleotides. Alternatively, substitution of pyrimidine nucleotides by modified analogues, e.g., substitution of uridine by 2′-deoxythymidinc is tolerated and does not affect the efficiency of RNA interference.

In a preferred aspect, the invention features RNA silencing agents that include first and second strands wherein the second strand and/or first strand is modified by the substitution of internal nucleotides with modified nucleotides, such that in vivo stability is enhanced as compared to a corresponding unmodified RNA silencing agent. As defined herein, an “internal” nucleotide is one occurring at any position other than the 5′ end or 3′ end of nucleic acid molecule, polynucleotide or oligonucleotide. An internal nucleotide can be within a single-stranded molecule or within a strand of a duplex or double-stranded molecule. In one embodiment, the sense strand and/or antisense strand is modified by the substitution of at least one internal nucleotide. In another embodiment, the sense strand and/or antisense strand is modified by the substitution of at least 2, 3, 4, 5, 6, 7, 8, 9, 10, 11, 12, 13, 14, 15, 16, 17, 18, 19, 20, 21, 22, 23, 24, 25 or more internal nucleotides. In another embodiment, the sense strand and/or antisense strand is modified by the substitution of at least 5%, 10%, 15%, 20%, 25%, 30%, 35%, 40%, 45%, 50%, 55%, 60%, 65%, 70%, 75%, 80%, 85%, 90%, 95% or more of the internal nucleotides. In yet another embodiment, the sense strand and/or antisense strand is modified by the substitution of all of the internal nucleotides.

In a preferred embodiment of the present invention, the RNA silencing agents may contain at least one modified nucleotide analogue. The nucleotide analogues may be located at positions where the target-specific silencing activity, e.g., the RNAi mediating activity or translational repression activity is not substantially effected, e.g., in a region at the 5′-end and/or the 3′-end of the siRNA molecule. Particularly, the ends may be stabilized by incorporating modified nucleotide analogues.

Exemplary nucleotide analogues include sugar-and/or backbone-modified ribonucleotides (i.e., include modifications to the phosphate-sugar backbone). For example, the phosphodiester linkages of natural RNA may be modified to include at least one of a nitrogen or sulfur heteroatom. In exemplary backbone-modified ribonucleotides, the phosphoester group connecting to adjacent ribonucleotides is replaced by a modified group, e.g., of phosphothioate group. In exemplary sugar-modified ribonucleotides, the 2′ OH-group is replaced by a group selected from H, OR, R, halo, SH, SR, NH₂, NHR, NR₂ or ON, wherein R is C₁-C₆ alkyl, alkenyl or alkynyl and halo is F, Cl, Br or I.

In particular embodiments, the modifications are 2′-fluoro, 2′-amino and/or 2′-thio modifications. Particularly preferred modifications include 2′-fluoro-cytidine, 2′-fluoro-uridine, 2′-fluoro-adenosine, 2′-fluoro-guanosine, 2′-amino-cytidine, 2′-amino-uridine, 2′-amino-adenosine, 2′-amino-guanosine, 2,6-diaminopurine, 4-thio-uridine, and/or 5-amino-allyl-uridine. In a particular embodiment, the 2′-fluoro ribonucleotides are every uridine and cytidine. Additional exemplary modifications include 5-bromo-uridine, 5-iodo-uridine, 5-methyl-cytidine, ribo-thymidine, 2-aminopurine, 2′-amino-butyryl-pyrene-uridine, 5-fluoro-cytidine, and 5-fluoro-uridine. 2′-deoxy-nucleotides and 2′-Ome nucleotides can also be used within modified RNA-silencing agents moities of the instant invention. Additional modified residues include, deoxy-abasic, inosine, N3-methyl-uridine, N6,N6-dimethyl-adenosine, pseudouridine, purine ribonucleoside and ribavirin. In a particularly preferred embodiment, the 2′ moiety is a methyl group such that the linking moiety is a 2′-O-methyl oligonucleotide.

In an exemplary embodiment, the RNA silencing agent of the invention comprises Locked Nucleic Acids (LNAs). LNAs comprise sugar-modified nucleotides that resist nuclease activities (are highly stable) and possess single nucleotide discrimination for mRNA (Elmen et al., Nucleic Acids Res., (2005), 33 (1): 439-447; Braasch et al. (2003) Biochemistry 42:7967-7975, Petersen et al. (2003) Trends Biotechnol 21:74-81). These molecules have 2′-0,4′-C-ethylene-bridged nucleic acids, with possible modifications such as 2′-deoxy-2″-fluorouridine. Moreover, LNAs increase the specificity of oligonucleotides by constraining the sugar moiety into the 3′-endo conformation, thereby pre-organizing the nucleotide for base pairing and increasing the melting temperature of the oligonucleotide by as much as 10° C. per base.

In another exemplary embodiment, the RNA silencing agent of the invention comprises Peptide Nucleic Acids (PNAs). PNAs comprise modified nucleotides in which the sugar-phosphate portion of the nucleotide is replaced with a neutral 2-amino ethylglycine moiety capable of forming a polyamide backbone which is highly resistant to nuclease digestion and imparts improved binding specificity to the molecule (Nielsen, et al., Science, (2001), 254:1497-1500).

Also preferred are nucleobase-modified ribonucleotides, i.e., ribonucleotides, containing at least one non-naturally occurring nucleobase instead of a naturally occurring nucleobase. Bases may be modified to block the activity of adenosine deaminase. Exemplary modified nucleobases include, but are not limited to, uridine and/or cytidine modified at the 5-position, e.g., 5-(2-amino) propyl uridine, 5-bromo uridine; adenosine and/or guanosines modified at the 8 position, e.g., 8-bromo guanosine; deaza nucleotides, e.g., 7-deaza-adenosine; O-and N-alkylated nucleotides, e.g., N6-methyl adenosine are suitable. It should be noted that the above modifications may be combined.

In other embodiments, cross-linking can be employed to alter the pharmacokinetics of the RNA silencing agent, for example, to increase half-life in the body. Thus, the invention includes RNA silencing agents having two complementary strands of nucleic acid, wherein the two strands are crosslinked. The invention also includes RNA silencing agents which are conjugated or unconjugated (e.g., at its 3′ terminus) to another moiety (e.g., a non-nucleic acid moiety such as a peptide), an organic compound (e.g., a dye), or the like). Modifying siRNA derivatives in this way may improve cellular uptake or enhance cellular targeting activities of the resulting siRNA derivative as compared to the corresponding siRNA, are useful for tracing the siRNA derivative in the cell or improve the stability of the siRNA derivative compared to the corresponding siRNA.

Other exemplary modifications include: (a) 2′ modification, e.g., provision of a 2′ OMe moicty on a U in a sense or antisense strand, but especially on a sense strand, or provision of a 2′ OMe moiety in a 3′ overhang, e.g., at the 3′ terminus (3′ terminus means at the 3′ atom of the molecule or at the most 3′ moiety, e.g., the most 3′ P or 2′ position, as indicated by the context); (b) modification of the backbone, e.g., with the replacement of an 0 with an S, in the phosphate backbone, e.g., the provision of a phosphorothioate modification, on the U or the A or both, especially on an antisense strand; e.g., with the replacement of a P with an S; (c) replacement of the U with a C5 amino linker; (d) replacement of an A with a G (sequence changes are preferred to be located on the sense strand and not the antisense strand); and (d) modification at the 2′, 6′, 7′, or 8′ position. Exemplary embodiments are those in which one or more of these modifications are present on the sense but not the antisense strand, or embodiments where the antisense strand has fewer of such modifications. Yet other exemplary modifications include the use of a methylated P in a 3′ overhang, e.g., at the 3′ terminus; combination of a 2′ modification, e.g., provision of a 2′ O Me moiety and modification of the backbone, e.g., with the replacement of a P with an S, e.g., the provision of a phosphorothioate modification, or the use of a methylated P, in a 3′ overhang, e.g., at the 3′ terminus; modification with a 3′ alkyl; modification with an abasic pyrrolidone in a 3′ overhang, e.g., at the 3′ terminus; modification with naproxen, ibuprofen, or other moieties which inhibit degradation at the 3′ terminus.

### Oligonucleotide Conjugate

The oligonucleotide conjugates described here comprise an oligonucleotide linked to a functional moiety.

Each of the functional moieties described above are depicted below structurally.

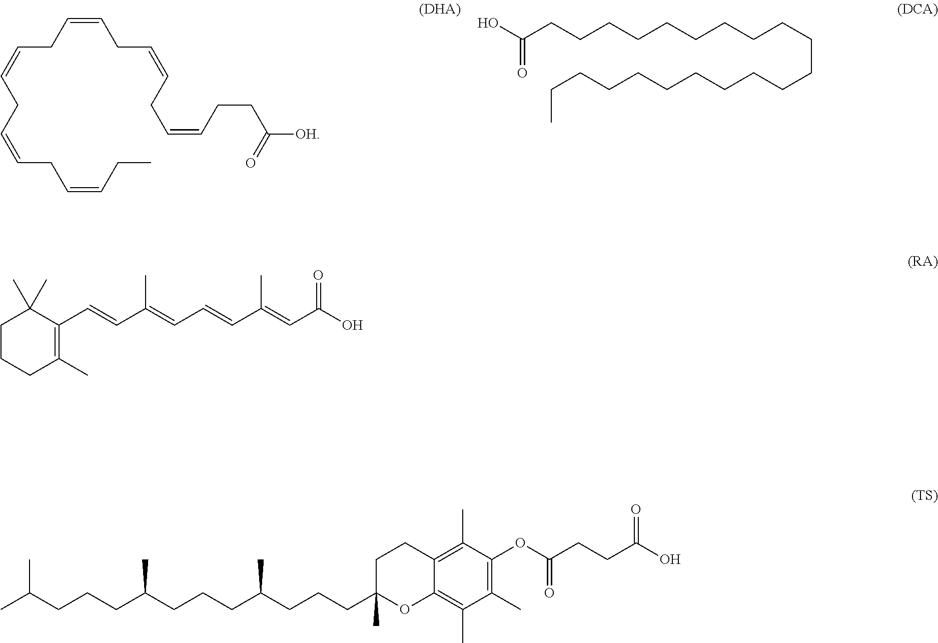 [SMILES: CC/C=C\C/C=C\C/C=C\C/C=C\C/C=C\C/C=C\CCC(=O)O ; CCCCCCCCCCCCCCCCCCCCCC(=O)O ; CC1=C(/C=C/C(C)=C/C=C/C(C)=C/C(=O)O)C(C)(C)CCC1 ; Cc1c(C)c2c(c(C)c1OC(=O)CCC(=O)O)CC[C@@](C)(CCC[C@H](C)CCC[C@H](C)CCCC(C)C)O2]

In certain embodiments, two DHA functional moieties are linked to the oligonucleotide.

In certain embodiments, the oligonucleotide comprises an antisense oligonucleotide or an siRNA.

In certain embodiments, the siRNA comprises a sense strand and an antisense strand. In certain embodiments, the antisense strand comprises about 15 nucleotides to 25 nucleotides in length (e.g., 15, 16, 17, 18, 19, 20, 21, 22, 23, 24, or 25 nucleotides in length). In certain embodiments, the antisense strand is 20 nucleotides in length, 21 nucleotides in length, or 22 nucleotides in length. In certain embodiments, the sense strand comprises about 15 nucleotides to 25 nucleotides in length (e.g., 15, 16, 17, 18, 19, 20, 21, 22, 23, 24, or 25 nucleotides in length). In certain embodiments, the sense strand is 15 nucleotides in length, 16 nucleotides in length, 18 nucleotides in length, or 20 nucleotides in length.

In certain embodiments, the siRNA comprises a double-stranded region of 15 base pairs to 20 base pairs (e.g., 15, 16, 17, 18, 19, or 20 base pairs). In certain embodiments, the siRNA comprises a double-stranded region of 15 base pairs, 16 base pairs, 18 base pairs, or 20 base pairs.

In certain embodiments, the siRNA comprises at least one blunt-end. In certain embodiments, the siRNA comprises two blunt-ends.

In certain embodiments, the siRNA comprises at least one single stranded nucleotide overhang (also referred to herein as a “single-stranded tail”). In certain embodiments, the siRNA comprises two single stranded nucleotide overhangs. In certain embodiments, the siRNA comprises about a 2-nucleotide to 5-nucleotide single stranded nucleotide overhang (e.g., a 2-, 3-, 4-, or 5-nucleotide overhang). In certain embodiments, the siRNA comprises a 2-nucleotide single stranded nucleotide overhang or a 5-nucleotide single stranded nucleotide overhang.

In certain embodiments, the siRNA comprises naturally occurring nucleotides (i.e., unmodified ribonucleotides).

In certain embodiments, the siRNA comprises at least one modified nucleotide. In certain embodiments, the modified nucleotide comprises a 2′-O-methyl modified nucleotide, a 2′-deoxy-2′-fluoro modified nucleotide, a 2′-deoxy-modified nucleotide, a locked nucleotide, an abasic nucleotide, a 2′-amino-modified nucleotide, a 2′-alkyl-modified nucleotide, a morpholino nucleotide, a phosphoramidate, a non-natural base comprising nucleotide, or a mixture thereof.

In certain embodiments, the siRNA comprises at least one modified internucleotide linkage. In certain embodiments, the modified internucleotide linkage comprises a phosphorothioate internucleotide linkage. In certain embodiments, the siRNA comprises 4-16 phosphorothioate internucleotide linkages. In certain embodiments, the siRNA comprises 8-13 phosphorothioate internucleotide linkages.

In certain embodiments, the siRNA comprises at least 80% chemically modified nucleotides (e.g., 80%, 81%, 82%, 83%, 84%, 85%, 86%, 87%, 88%, 89%, 90%, 91%, 92%, 93%, 94%, 95%, 96%, 97%, 98%, 99%, or 100% chemically modified nucleotides). In certain embodiments, the siRNA is fully chemically modified.

In certain embodiments, the siRNA comprises at least 70% 2′-O-methyl nucleotide modifications (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% % 2′-O-methyl nucleotide modifications). In certain embodiments, the antisense strand comprises at least 70% 2′-O-methyl nucleotide modifications (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% % 2′-O-methyl nucleotide modifications). In certain embodiments, the antisense strand comprises about 70% to 90% 2′-O-methyl nucleotide modifications. In certain embodiments, the sense strand comprises at least 65% 2′-O-methyl nucleotide modifications (e.g., 65%, 66%, 67%, 68%, 69% 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% % 2′-O-methyl nucleotide modifications). In certain embodiments, the sense strand comprises 100% 2′-O-methyl nucleotide modifications.

In certain embodiments, the sense strand comprises one or more nucleotide mismatches between the antisense strand and the sense strand.

In certain embodiments, the antisense strand comprises a 5′ phosphate, a 5′-alkyl phosphonate, a 5′ alkylene phosphonate, or a 5′ alkenyl phosphonate. In certain embodiments, the antisense strand comprises a 5′ vinyl phosphonate.

In certain embodiments, the functional moiety is linked to the 5′ end and/or 3′ end of the oligonucleotide. In certain embodiments, the functional moiety is linked to the 5′ end and/or 3′ end of the sense strand or to the 5′ end and/or 3′ end of the antisense strand. In certain embodiments, the functional moiety is linked to the 3′ end of the sense strand.

In certain embodiments, the functional moiety is linked to the antisense strand and/or sense strand by a linker.

In certain embodiments, the linker comprises a divalent or trivalent linker.

In certain embodiments, the divalent or trivalent linker is selected from the group consisting of:

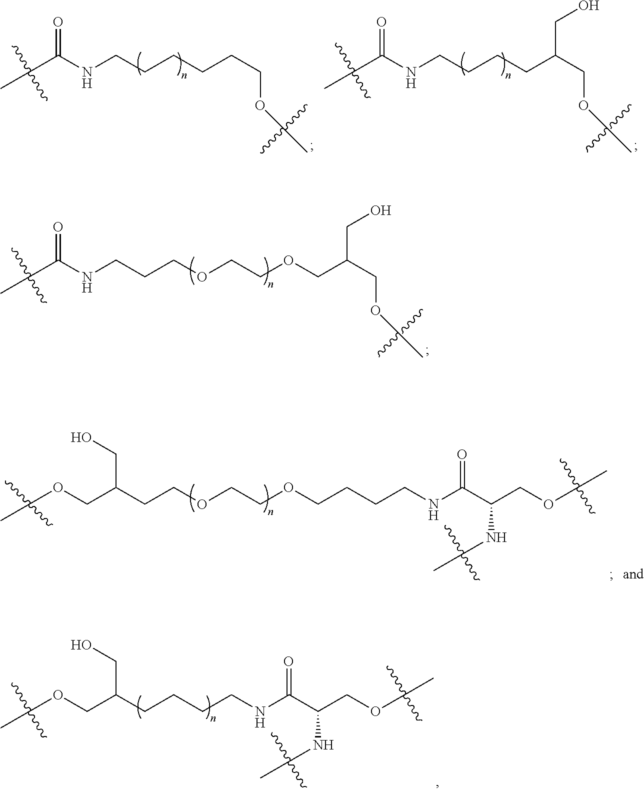

wherein n is 1, 2, 3, 4, or 5.

In certain embodiments, the linker comprises an ethylene glycol chain, an alkyl chain, a peptide, an RNA, a DNA, a phosphodiester, a phosphorothioate, a phosphoramidate, an amide, a carbamate, or a combination thereof.

In certain embodiments, when the linker is a trivalent linker, the linker further links a phosphodiester or phosphodiester derivative.

In certain embodiments, the phosphodiester or phosphodiester derivative is selected from the group consisting of:

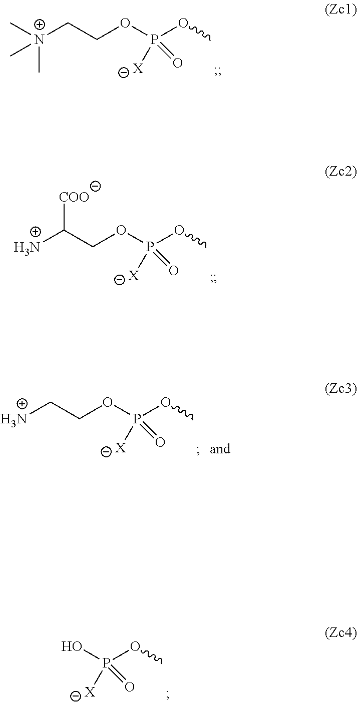

wherein X is O, S or BH₃.

The above recited moiety Zcl is phosphatidylcholine (PC). Any one of the functional moieties described herein may comprise a phosphatidylcholine (PC) esterified derivative, i.e., phosphatidylcholine (PC) esterified retinoic acid (PC-RA), phosphatidylcholine (PC) esterified docosahexaenoic acid (PC-DHA), phosphatidylcholine (PC) esterified docosanoic acid (PC-DCA), or phosphatidylcholine (PC) esterified a-tocopheryl succinate (PC-TS).

In certain embodiments, the nucleotides at positions 1 and 2 from the 3′ end of sense strand, and the nucleotides at positions 1 and 2 from the 5′ end of antisense strand are connected to adjacent ribonucleotides via phosphorothioate linkages.

In certain embodiments, the oligonucleotide conjugate comprises the structure:

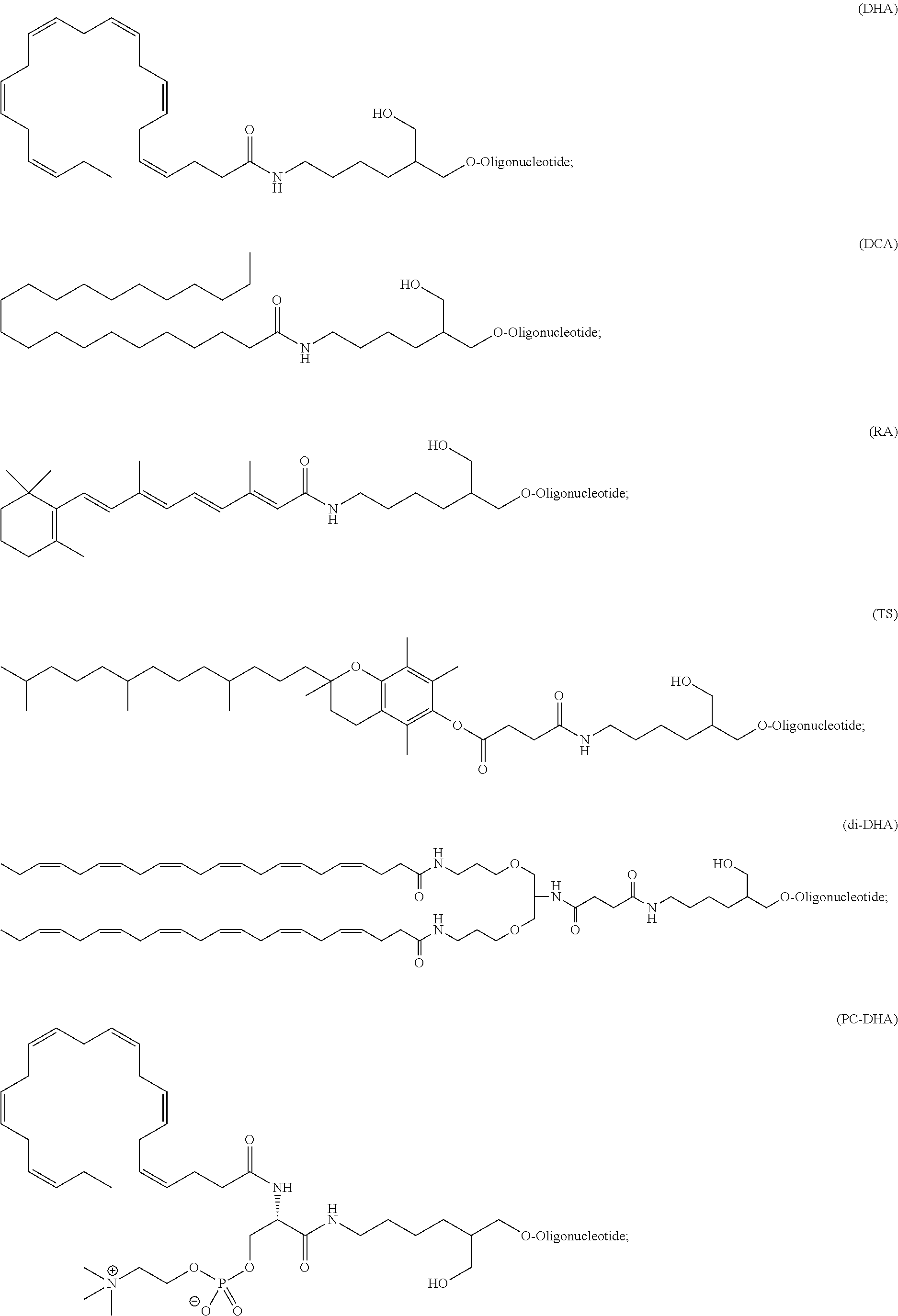

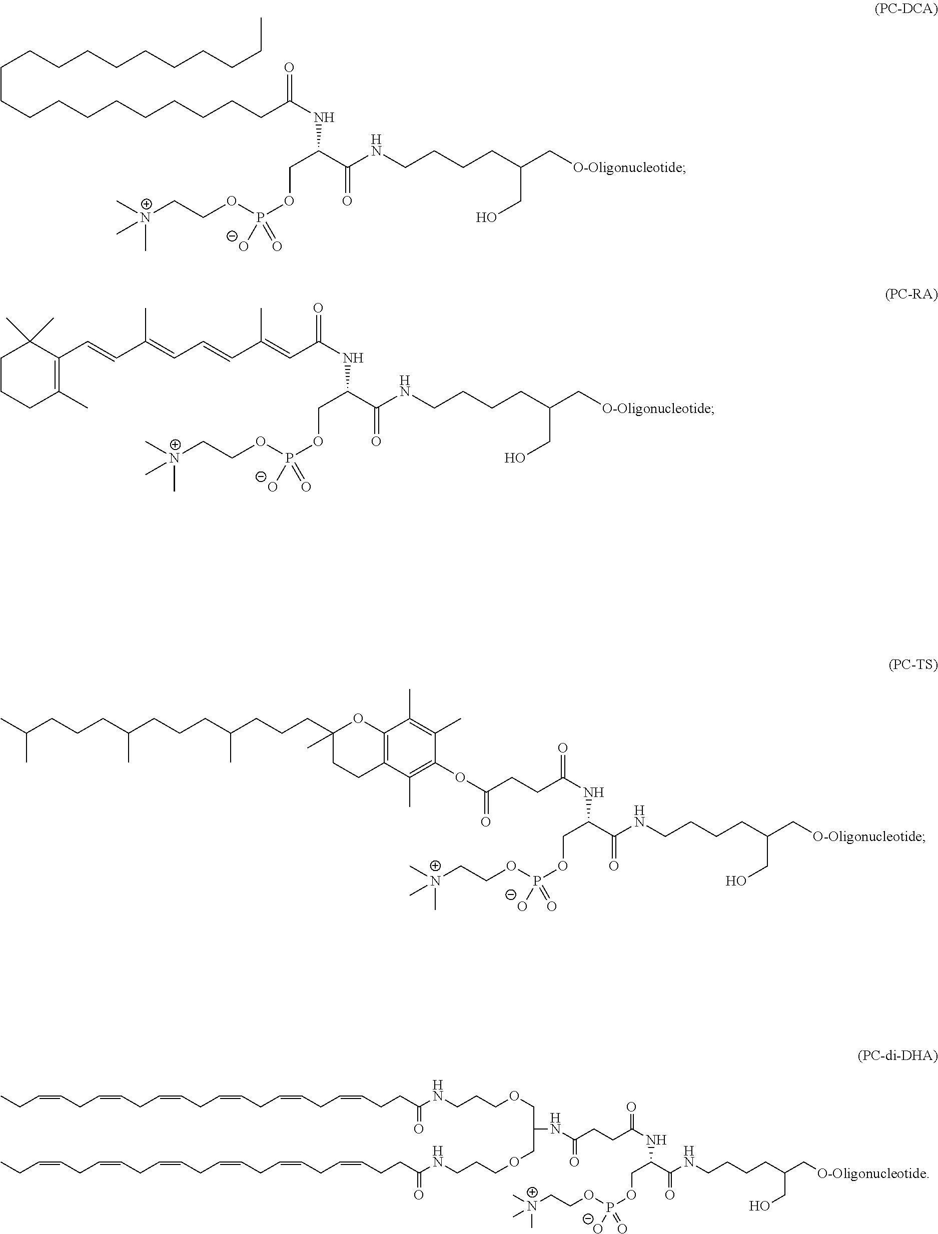

For any of the above recited structures, the term “oligonucleotide” corresponds to any of the oligonucleotides recited herein. e.g., an ASO or siRNA. In certain embodiments, the term “oligonucleotide” in the structures recited above corresponds to the sense strand of an siRNA. In certain embodiments, the oxygen immediately adjacent to the term “oligonucleotide” in the structures is linked to the 3′ end of a sense strand of an siRNA.

### Branched Oligonucleotides

The branched oligonucleotides described here comprise two or more oligonucleotides linked together. The different branched oligonucleotides described herein (e.g., a branched oligonucleotide with two, three, or four oligonucleotides) enhanced topical delivery of the oligonucleotide.

In certain embodiments, one or more of the oligonucleotides of the branched oligonucleotide further comprises a functional moiety linked to the oligonucleotide, wherein the functional moiety comprises any one of retinoic acid, DHA, DCA, or a-tocopheryl succinate. The functional moieties as described above in the Oligonucleotide Conjugate section can be applied to the oligonucleotides of the branched oligonucleotides. Similarly, the oligonucleotides as described above in the Oligonucleotide Conjugate section can serve as the oligonucleotides of the branched oligonucleotides, including type (ASO or siRNA), strand length, and chemical modifications.

In certain embodiments, the two or more oligonucleotides in the branched oligonucleotide are connected to one another by one or more moieties independently selected from a linker, a spacer and a branching point.

In certain embodiments, the linker comprises an ethylene glycol chain, an alkyl chain, a peptide, an RNA, a DNA, a phosphate, a phosphonate, a phosphoramidate, an ester, an amide, a triazole, or combinations thereof.

In certain embodiments, the branching point comprises a polyvalent organic species or derivative thereof.

In another embodiment, the branching point is an amino acid derivative. In another embodiment of the branching point is selected from the formulas of:

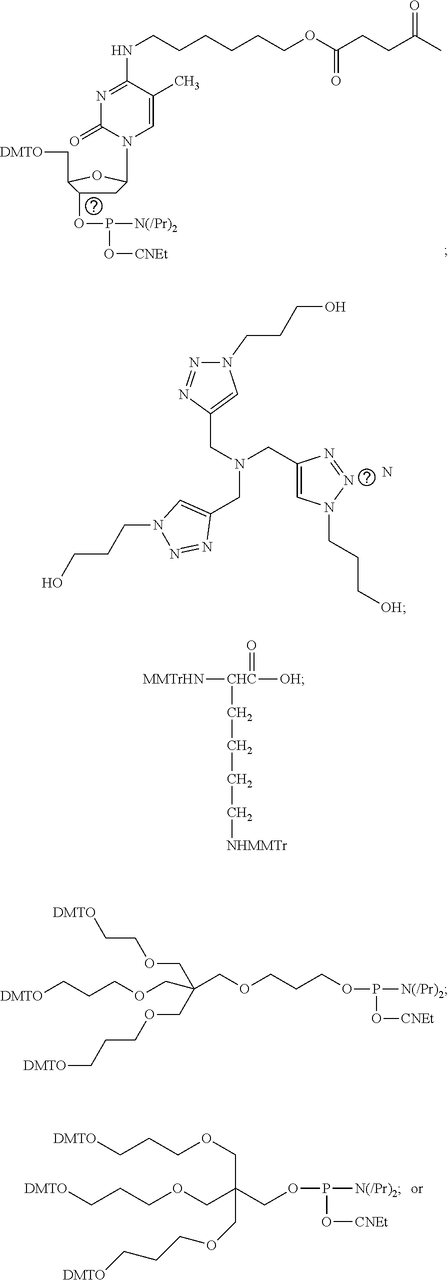

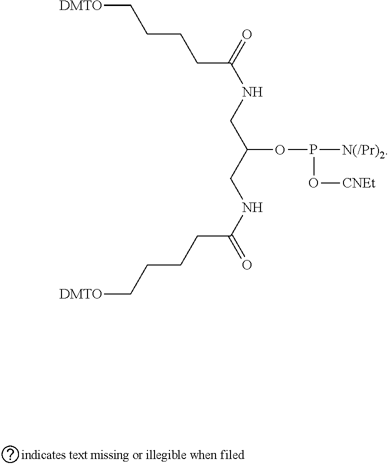

Polyvalent organic species are moieties comprising carbon and three or more valencies (i.e., points of attachment with moieties such as S, L or N, as defined above). Non-limiting examples of polyvalent organic species include triols (e.g., glycerol, phloroglucinol, and the like), tetrols (e.g., ribose, pentaerythritol, 1,2,3,5-tetrahydroxybenzene, and the like), tri-carboxylic acids (e.g., citric acid, 1,3,5-cyclohexanetricarboxylic acid, trimesic acid, and the like), tetra-carboxylic acids (e.g., ethylenediaminetetraacetic acid, pyromellitic acid, and the like), tertiary amines (e.g., tripropargylamine, triethanolamine, and the like), triamines (e.g., diethylenetriamine and the like), tetramines, and species comprising a combination of hydroxyl, thiol, amino, and/or carboxyl moieties (e.g., amino acids such as lysine, serine, cysteine, and the like).

In certain embodiments, the spacer comprises an ethylene glycol chain, an alkyl chain, a peptide, an RNA, a DNA, a phosphate, a phosphonate, a phosphoramidate, an ester, an amide, a triazole, or a combination thereof.

In certain embodiments, the linker comprises the structure L1:

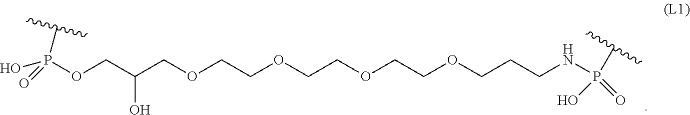 [SMILES: *P(=O)(O)NCCCOCCOCCOCCOCC(O)COP(*)(=O)O]

In certain embodiments, the linker comprises the structure L2:

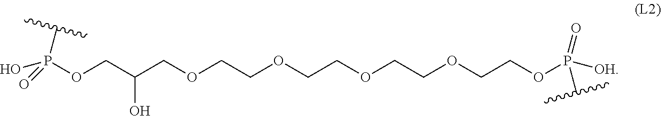 [SMILES: *P(=O)(O)OCCOCCOCCOCCOCC(O)COP(*)(=O)O]

In certain embodiments, the branched oligonucleotide consists of two oligonucleotides. In certain embodiments, the branched oligonucleotide consists of three oligonucleotides. In certain embodiments, the branched oligonucleotide consists of four oligonucleotides. In certain embodiments, the oligonucleotides are siRNA.

In certain embodiments, the branched oligonucleotide comprises the structure:

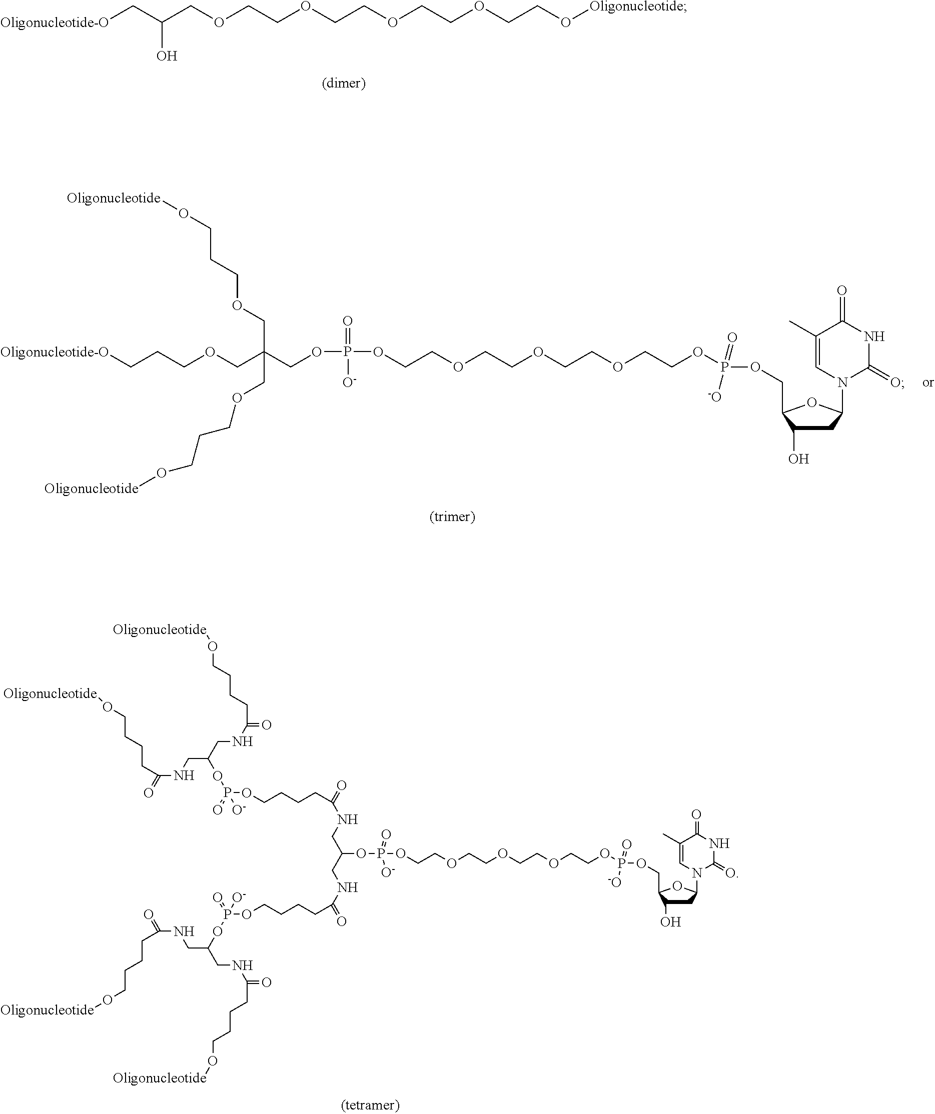

For any of the above recited structures, the term “oligonucleotide” corresponds to any of the oligonucleotides recited herein, e.g., an ASO or siRNA. In certain embodiments, the term “oligonucleotide” in the structures recited above corresponds to the sense strand of an siRNA. In certain embodiments, the oxygen immediately adjacent to the term “oligonucleotide” in the structures is linked to the 3′ end of a sense strand of an siRNA.

Branched oligonucleotides, including synthesis and methods of use, are described in greater detail in WO2017/132669, incorporated herein by reference.

### Dendron Conjugates

The oligonucleotide conjugates described herein comprise an oligonucleotide and a dendron. The dendron can have a strong reversible and non-covalent albumin binding that can be advantageously used to minimize degradation, reduce their uptake and degradation by macrophages, prevent non-specific uptake by cells, and/or provide enhanced delivery to skin of the oligonucleotide conjugate. The dendron can also have a nanomolar affinity to albumin that advantageously ensures that the albumin-oligonucleotide conjugate complex remains stable throughout biodistribution, cell interactions, and/or treatment.

In certain embodiments, the dendron comprises a hydrophilic end group, a phosphate group, and/or a hydrophobic chain.

In certain embodiments, the end group is a hydrophilic group comprising hydroxide, amine, phosphate, sulfur, and/or sugar; an hydrophobic group comprising amine, amide, ether, ester, N -or O-containing heterocycles, thiol, thioether, and/or saturated or unsaturated C₁₋₂₄ alkyl chain; and/or one or more aromatic rings.

In certain embodiments, the hydrophobic chain is a saturated or unsaturated C₁₋₂₄ alkyl group.

In certain embodiments, the dendron comprises two branches. In certain embodiments, the dendron comprises four branches. In certain embodiments, the dendron comprises eight branches.

In certain embodiments, the oligonucleotide conjugate has a structure of Formula I:

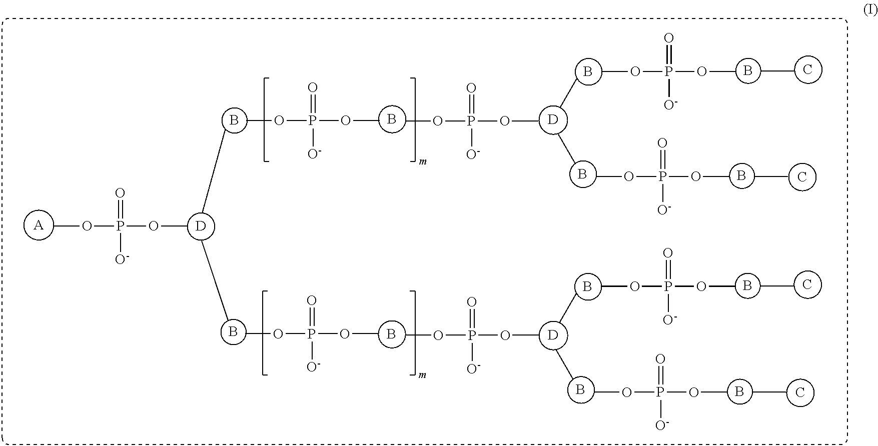

wherein: A is an oligonucleotide; B, for each occurrence, independently comprises one or more hydrophobic chains, amines, amides, esters, an N -or O-containing heterocycles, thioethers, disulfides, and/or aromatic rings, wherein the hydrophobic chain comprises a saturated or unsaturated C₁₋₂₄ alkyl chain; C, for each occurrence, independently comprises: an hydrophilic group comprising hydroxide, amine, phosphate, sulfur, and/or sugar; an hydrophobic group comprising amine, amide, ether, ester, N -or O-containing heterocycles, thiol, thioether, and/or saturated or unsaturated C₁₋₂₄ alkyl chain; and/or one or more aromatic rings; D, for each occurrence, independently is a branching unit comprising one or more alkyl chains, amides, ethers, esters, and amines, wherein the branching unit comprises 2 to 4 branches; and m, for each occurrence, independently is 0 or 1.

In certain embodiments, the oligonucleotide conjugate has a structure of Formula II:

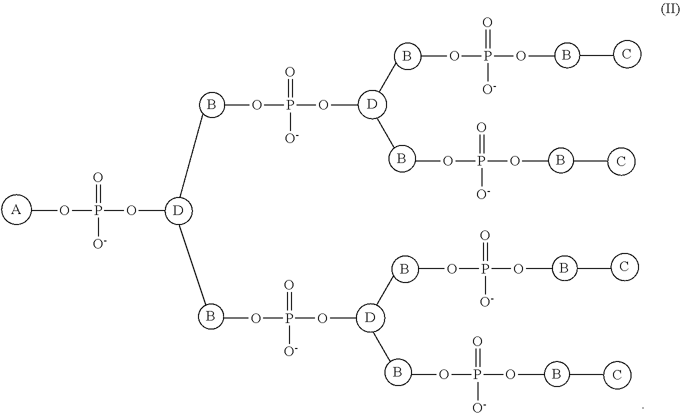

In certain embodiments, the oligonucleotide conjugate has a structure of Formula III:

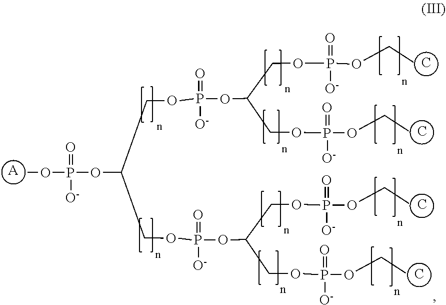

wherein n, for each occurrence, independently is an integer between 1-24.

In certain embodiments, C is OH and n, for each occurrence, independently is 1, 6, or 12.

In certain embodiments, the oligonucleotide conjugate has the structure of Formula IV:

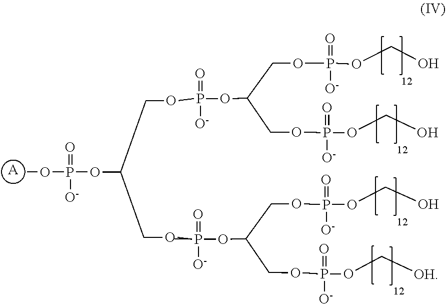

In certain embodiments, the oligonucleotide conjugate has a structure of Formula V:

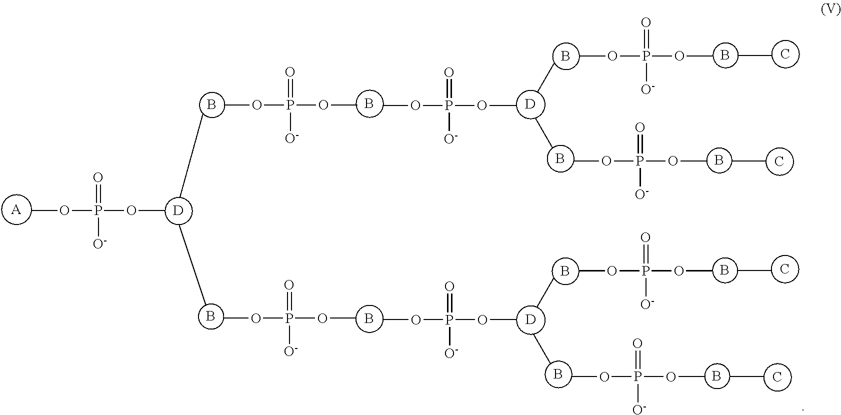

In certain embodiments, the oligonucleotide conjugate has a structure of Formula VI:

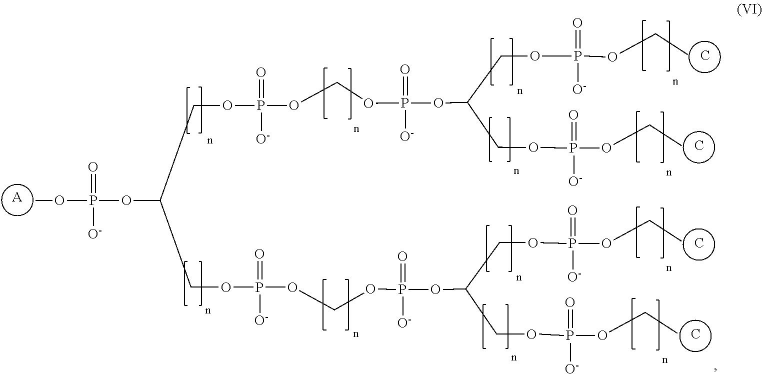

wherein n, for each occurrence, independently is an integer between 1-24.

In certain embodiments, C is OH and n, for each occurrence, independently is 1, 6, or 12.

In certain embodiments, the oligonucleotide conjugate has the structure of Formula VII

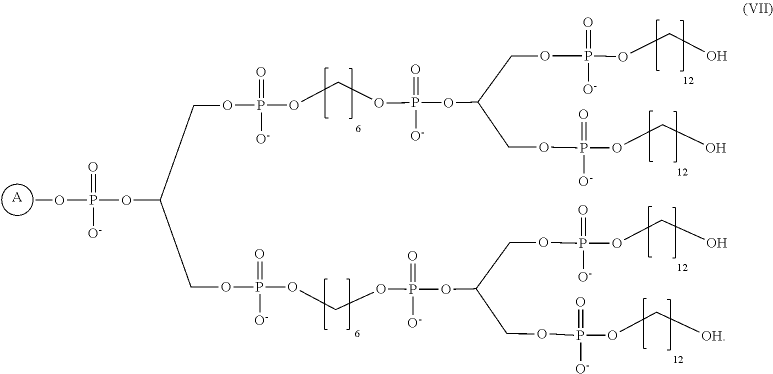

### Branched Oligonucleotide Conjugate

In certain embodiments, the oligonucleotide conjugate is a branched oligonucleotide conjugate.

In certain embodiments, the linker comprises an ethylene glycol chain, an alkyl chain, a peptide, an RNA, a DNA, a phosphate, a phosphonate, a phosphoramidate, an ester, an amide, a triazole, or any combinations thereof.

In certain embodiments, the branching point comprises a polyvalent organic species or derivative thereof.

In another embodiment, the branching point is an amino acid derivative. In another embodiment of the branching point is selected from the formulas of:

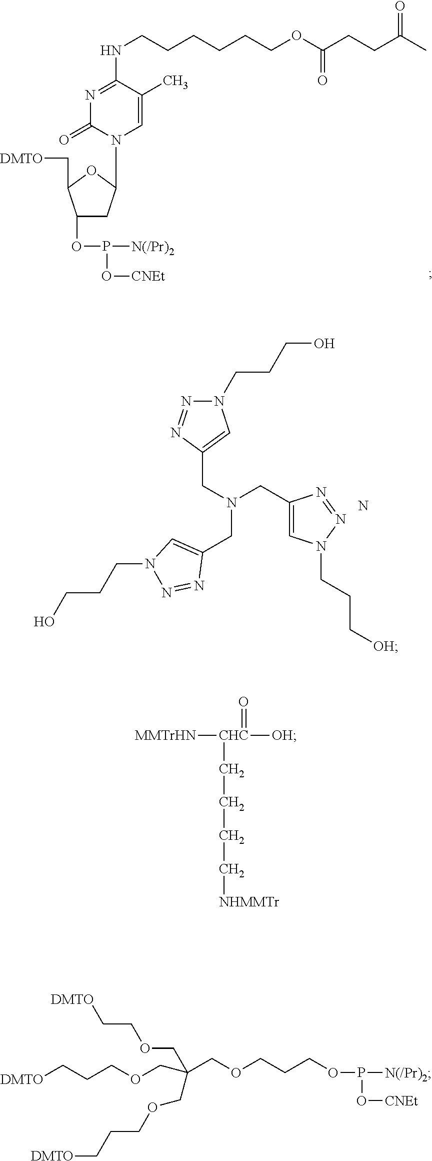

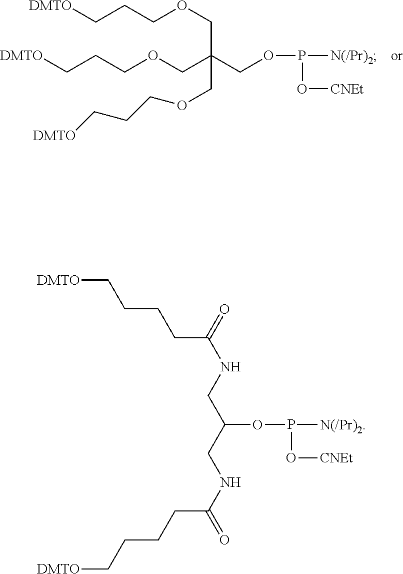

Polyvalent organic species are moieties comprising carbon and three or more valencies (i.e., points of attachment with moieties such as S, L or N, as defined above). Non-limiting examples of polyvalent organic species include triols (e.g., glycerol, phloroglucinol, and the like), tetrols (e.g., ribose, pentaerythritol, 1,2,3,5-tetrahydroxybenzene, and the like), tri-carboxylic acids (e.g., citric acid, 1,3,5-cyclohexanetricarboxylic acid, trimesic acid, and the like), tetra-carboxylic acids (e.g., ethylenediaminetetraacetic acid, pyromellitic acid, and the like), tertiary amines (e.g., tripropargylamine, triethanolamine, and the like), triamines (e.g., diethylenetriamine and the like), tetramines, and species comprising a combination of hydroxyl, thiol, amino, and/or carboxyl moieties (e.g., amino acids such as lysine, serine, cysteine, and the like).

In certain embodiments, the spacer comprises an ethylene glycol chain, an alkyl chain, a peptide, an RNA, a DNA, a phosphate, a phosphonate, a phosphoramidate, an ester, an amide, a triazole, or a combination thereof.

### Microneedle Particles

The microneedle particles are structured to at least partially penetrate a biological tissue, such as the stratum corneum of human skin. That is, the microneedle portions (or at least the tip end portion thereof) of the particles are dimensioned and possess the mechanical rigidity to enable them to be pressed into and penetrate the biological tissue, forming a microscale hole or channel therein, and the microneedle particles each have an overall geometric shape or other design feature that generally prevents the particle as a whole from penetrating into the biological tissue. Mere elastic deformation of the biological tissue is not penetration; penetration may include elastic deformation, but further includes penetration into the tissue.

In some embodiments, this advantageously may facilitate removal of the microneedle particles from the surface of the biological tissue after the desired microchannels are formed therein. For example, after at least partially penetrating a biological tissue, the microneedle particles may be washed or wiped away from the biological tissue, where the microneedle particles are configured to not become completely and/or irremovably embedded in the biological tissue.

In some other embodiments, however, the microneedle particles are configured to prevent the entire structure of the microneedle particles from penetrating a biological tissue, and the microneedle particles have one or more structural features that prevent their removal from the biological tissue and/or that increases the difficulty of removing the microneedle particles from the biological tissue.

In embodiments, the microneedle particles include a core structure and one or more microneedles extending from the core structure. The one or more microneedles may be structured to at least partially penetrate a biological tissue. For example, the one or more microneedles may be structured to at least partially penetrate a biological tissue to form a microchannel in the biological tissue. The one or more microneedles may extend independently in any direction from the core structure. In one embodiment, the one or more microneedles are structured to [1] at least partially penetrate a first type of biological tissue, and [2] prevent or decrease the likelihood that the one or more microneedles can penetrate a second type of biological tissue. The second type of biological tissue, for example, may include the skin of the fingers, while the first type of biological tissue may include a tissue to be treated, for example, an area of the skin having a relatively thinner stratum corneum or a mucosal tissue. In this way, for example, the one or more microneedles may not penetrate, or may be less likely to penetrate, the skin of the fingers used to apply or rub the microneedle particles onto/into the treatment area of the first biological tissue.

In some embodiments, the microneedle particles comprise one microneedle, two microneedles, three microneedles, four microneedles, five microneedles, six microneedles, seven microneedles, eight microneedles, nine microneedles, or ten microneedles extending from the core structure. In a preferred embodiment, the microneedle particles each have from 3 to 10 microneedles. In a particular embodiment, the microneedle particles comprise an odd number of microneedles; for example, one microneedle, three microneedles, five microneedles, seven microneedles, or nine microneedles. In a still further embodiment, the microneedle particles comprise an even number of microneedles; for example, two microneedles, four microneedles, six microneedles, eight microneedles, or ten microneedles. In another embodiment, the microneedle particles comprise 3 to 100 microneedles, 3 to 75 microneedles, 3 to 50 microneedles, 3 to 25 microneedles, 3 to 20 microneedles, or 10 to 20 microneedles.

The core structure typically is the portion of the microneedle particle that connects the microneedles, especially when there are three or more microneedles. When a microneedle particle has only two microneedles, the core structure consists of the portion of the microneedle particle that connects the two microneedles, or the interface between the two microneedles. When a microneedle particle has only one microneedle, the core structure can include the non-penetrating portion of the microneedle particle. Such a non-penetrating portion is provided at the base of the microneedle (distal to the tip) and would include a laterally extending portion (lateral with respect to the longitudinal axis of the microneedle) that is effective to function as a penetration stop. It may be shaped as a ball or a flange, for example.

The core structure may be a solid structure, or a hollow structure having one or more internal cavities. When the core structure has a hollow structure, the core structure may be filled with a material, which may be delivered to a biological tissue. The material, which may be a solid or liquid, may be or include a bioactive agent and/or other substance of interest. The core structure may be configured to expand/swell, which may permit the microneedle particles to be delivered and applied to an internal biological tissue, such as the gastrointestinal tract. Prior to the initiation of expansion/swelling, the one or more microneedles may not penetrate, or may be less likely to penetrate, a biological tissue. The core structure may be configured to expand/swell upon or after reaching a desired location, so that the one or more microneedles only penetrate, or are more likely to penetrate, a biological tissue at or near the desired location.

Various design features of the microneedle particles may be selected to impart the particles with the functionality preventing the entire microneedle particle from penetrating a biological tissue. These features may include the core structure itself, the one or more microneedles themselves, or the spatial relationship between/among the microneedles or a subset of those microneedles. Of course, a combination of these features may be designed to prevent the entire microneedle particle from penetrating a biological tissue.

For example, the core structure may have a size, shape, and/or a lack of sharp edges that permits one or more microneedles extending from the core structure to penetrate a biological tissue, but that inhibits all or substantially all of the core structure from penetrating into the biological tissue. As a further example, the one or more microneedles may have a structural feature, such as tapering, that permits only a portion of the one or more microneedles to penetrate a biological tissue. For example, a microneedle may have a shoulder or plateau that permits only the portion of the microneedle below the shoulder or plateau to penetrate the biological tissue. Such a configuration may prevent the core structure from contacting the biological tissue. As yet another example, two or more microneedles may be spatially arranged with respect to one another so that as one of the microneedles penetrates a biological tissue, the other microneedle(s) is/are fixed in an orientation that impart(s) resistance to further penetration by the particle, preventing the entire microneedle particle from penetrating the biological tissue. For example, one microneedle may be oriented toward and into the biological tissue, while one or more other microneedles of the particle extend in a lateral orientation, so that the flat sidewall of at least one of the other microneedles faces the biological tissue. The resistance may be provided when any part of the other microneedle(s) contact(s) the biological tissue. Due at least in part to the fact that the microneedle particle may be configured to prevent the entire microneedle particle from penetrating a tissue surface, a substance of interest delivered by the microneedle particle may be associated with the microneedle particle in a manner and/or at a position that ensures or increases the likelihood that at least a portion of the substance of interest [1] will enter a tissue surface and/or be released beneath a tissue surface, [2] will not enter a tissue surface and/or be released on or above the tissue surface, or [3] a combination thereof.

The one or more microneedles may extend from the core structure in a manner that imparts a symmetrical structure to the microneedle particle. Alternatively, the one or more microneedles may extend from the core structure in a manner that imparts an asymmetrical structure to the microneedle particle. A particle may have an even number of microneedles or an odd number of microneedles.

Generally, the one or more microneedles of a microneedle particle can have the same or different dimensions. In one embodiment, the one or more microneedles of the microneedle particles have substantially the same dimensions. In another embodiment, the one or more microneedles of the microneedle particles have different dimensions. For example, a microneedle particle may have four microneedles, and [1] all four microneedles may have the same dimensions, [2] all four microneedles may have different dimensions, [3] a first pair of the four microneedles may have the same dimensions, and those dimensions may differ from those of the second pair of the four microneedles, and the second pair of the four microneedles can include two microneedles having the same or different dimensions, [4] three of the four microneedles may have the same dimensions, and those dimensions may differ from the dimensions of the fourth microneedle.

The one or more microneedles may have any shape capable of at least partially penetrating a biological tissue. In embodiments, the one or more microneedles are high-aspect-ratio structures having a length at least two times greater than a width. The length of a microneedle is the distance from the core structure to the tip of the microneedle. In one embodiment, each of the one or more microneedles independently has a length of about 1 μm to about 2,000 μm. In one particular embodiment, each of the one or more microneedles independently has a length of about 10 μm to about 2,000 μm. In some embodiments, each of the one or more microneedles independently has a length of about 50 μm to about 2,000 μm. In another embodiment, each of the one or more microneedles independently has a length of about 100 μm to about 1,000 μm. In a further embodiment, each of the one or more microneedles independently has a length of about 250 μm to about 750 μm. In yet another embodiment, each of the one or more microneedles independently has a length of about 100 μm to about 500 μm. In a still further embodiment, each of the one or more microneedles has a length of about 500 μm. In particular embodiments, the microneedle particles comprise one microneedle, two microneedles, three microneedles, four microneedles, five microneedles, six microneedles, seven microneedles, eight microneedles, nine microneedles, or ten microneedles, and each of the microneedles independently has a length of about 1 μm to about 2,000 μm, about 10 μm to about 2,000 μm, about 50 μm to about 2,000 μm, about 100 μm to about 1,000 μm, or about 250 μm to about 750 μm.

In embodiments, at least one dimension of the one or more microneedles may be tapered. For example, one or more dimensions of the one or more microneedles, such as the width and/or height of the one or more microneedles may be greatest at a particular position, such as a position adjacent to the core structure.

The one or more microneedles may have a tip having a width of about 0.1 μm to about 30 μm. In embodiments, the one or more microneedles have a tip having a width of about 0.1 μm to about 30 μm, about 0.1 μm to about 25 μm, about 0.1 μm to about 20 μm, about 0.1 um to about 15 μm, about 0.1 μm to about 10 μm, about 0.1 μm to about 5 μm, about 1 μm to about 10 μm, about 1 μm to about 7 μm, about 1 μm to about 5 μm, about 1 μm to about 4 μm, or about 1 μm to about 3 μm. In one embodiment, each microneedle has a tip having a width of about 0.1 μm to about 5 μm. The “tip” typically is the portion of the one or more microneedles that first penetrates a biological tissue.

The microneedle particles generally may be of any size, including a size that prevents or reduces the likelihood of the microneedle particle becoming completely or irremovably embedded in the biological tissue. In one embodiment, the greatest dimension of the microneedle particles is about 10 μm to about 10,000 μm, 10 μm to about 5,000 μm, 100 μm to about 10,000 μm, about 250 μm to about 5,000 μm, about 500 to about 2,000 μm, or about 1,000 μm. The “greatest dimension of the microneedle particles” refers to the greatest of the following distances: [1] the distance between the tips of the two microneedles that are the farthest apart (if the microneedle particle includes two or more microneedles), or [2] the farther possible distance between a tip of a microneedle and the side of the core structure that is opposite the side from which the measured microneedle extends. A plurality of microneedle particles may include microneedle particles of one or more sizes.

In embodiments, the one or more microneedles are planar microneedles. The phrase “planar microneedles,” as used herein, refers to two or more microneedles, each having either [1] a central axis that extends from the core structure in at least substantially the same plane, or [2] a tip that exists in substantially the same plane. The planar microneedles may include microneedles that extend from the core structure in the same direction, different directions, or a combination thereof. The planar microneedles also may include co-linear planar microneedles, which extend from opposite sides of the core structure in a manner that permits the central axis of cach microneedle to at least substantially correspond with a single line.

In one embodiment, the microneedle particles have two planar microneedles, three planar microneedles, four planar microneedles, five planar microneedles, six planar microneedles, seven planar microneedles, eight planar microneedles, nine planar microneedles, or ten planar microneedles.

When the one or more microneedles are planar microneedles, the microneedle particles may have a substantially planar, i.e., flat, structure. The substantially planar, i.e., flat, microneedle particles may have a thickness of about 1 μm to about 1,000 μm, about 5 μm to about 500 μm, about 10 μm to about 250 μm, 50 μm to about 250 μm, about 50 μm to about 200 μm, about 75 μm to about 200 μm, about 75 μm to about 150 μm, about 75 μm to about 125 μm, or about 80 μm to about 120 μm. It was surprisingly discovered that the microneedle particles having a substantially planar, i.e., flat, structure, were effective in the methods provided herein, and that embodiments of the methods provided herein do not require the use of microneedle particles having a non-flat structure. Not wishing to be bound by any particular theory, it was believed that instead of sliding across tissue surfaces without penetrating the tissue surfaces, as expected, the microneedle particles provided herein were able to at least partially penetrate tissue surfaces due at least in part to the elastically deformable nature of the tissue surfaces, which can create “peaks” and “valleys” on the surface that microneedles can puncture when moving across the tissue surface. The tissue surfaces may be deformed, thereby facilitating penetration, by the one or more forces applied to the tissues by the microneedle particles.

When viewed in cross-section, the one or more microneedles may have a polygonal shape, a non-polygonal shape, a bisected non-polygonal shape, or a combination thereof. For example, a microneedle particle may include at least one microneedle having a non-polygonal shape when viewed in cross-section, and at least one microneedle having a polygonal shape when viewed in cross-section. As a further example, at least one microneedle of a microneedle particle may include at least one polygonal shape and at least one non-polygonal shape when viewed in cross-section at different positions. Non-limiting examples of non-polygonal shapes include circular, substantially circular, oval, and substantially oval. When a microneedle has a non-polygonal shape when viewed in cross-section, the microneedle may be at least substantially conical in shape, or at least a portion of the structure of the microneedle may be at least substantially conical. The phrase “bisected non-polygonal shape” refers to a polygonal shape that includes a bisecting flat surface. Non-limiting examples of bisected non-polygonal shapes include semi-circular, substantially semicircular, semi-oval, and substantially semi-oval. Non-limiting examples of polygonal shapes include triangular, square, rectangular, trapezoidal, diamond, pentagonal, hexagonal, septagonal, and octagonal. When a microneedle has a polygonal shape, such as triangular or square, the microneedle may be a pyramidal microneedle.

In embodiments, the one or more microneedles include non-planar microneedles. The phrase “non-planar microneedles,” as used herein, refers to microneedles having a central axis that extends from the core structure in different planes. In one embodiment, the one or more microneedles of the microneedle particle are non-planar microneedles. In another embodiment, the one or more microneedles include at least two planar microneedles, and at least one microneedle that is non-planar relative to the pair of planar microneedles. For example, a microneedle particle may have a substantially tetrahedral arrangement of four microneedles provided by two planar microneedles, and, relative to the two planar microneedles, two non-planar microneedles. As a further example, a microneedle particle may have a substantially octahedral arrangement of the one or more microneedles provided by four planar microneedles, and, relative to the four planar microneedles, two non-planar microneedles. As an additional example, a microneedle particle may have a group of planar microneedles, such as three, four, five, or six planar microneedles, and, relative to the group of planar microneedles, one non-planar microneedle.

In embodiments, at least one of the one or more microneedles is barbed. In other words, a microneedle of the particle may include one or more barbs. For example, a microneedle particle may include four microneedles, and one, two, three, or four of the microneedles may be barbed microneedles. A barbed microneedle generally is a microneedle having a structural feature rendering it difficult, if not impossible, in the absence of fracturing and/or eroding to remove the barbed microneedle from a biological tissue after the barbed microneedle at least partially penetrates the biological tissue. The barbed microneedle may include one or more projections angled away from the tip of the microneedle that at least partially penetrates a biological tissue. For example, a barbed microneedle may have a “fish hook” configuration in which one projection is angled away from the tip of the microneedle, or a barbed microneedle may have an “arrowhead” configuration in which two or more projections are angled away from the tip of the microneedle. The projections may have linear edges, curved edges, or a combination thereof.

In embodiments, the barbed microneedles that have at least partially penetrated a biological tissue may be configured to fracture upon removal of the microneedle particle from a biological tissue. Upon fracturing, at least a portion of the barbed microneedle may remain in and/or on the biological tissue. The portion of the barbed microneedle that remains in and/or on the biological tissue may include a substance of interest, such as a bioactive agent, which may include a pharmaceutical agent, a sensor, or a combination thereof. For example, the portion of the barbed microneedle that remains in and/or on the biological tissue may include a slow release formulation that releases a pharmaceutical agent during a desired dosage period. In one embodiment, the fracturing of the barbed microneedle occurs at or near the core structure. In another embodiment, the fracturing of the barbed microneedle occurs at or near the portion of the microneedle that is exposed to the greatest force when the barbed microneedle resists removal from the biological tissue. The barbed microneedles may include a predefined fracture region to ensure or at least increase the likelihood that the fracturing occurs at a desired location. The predefined fraction region may include a substantially narrowed portion, a perforated portion, a scored portion, a notched portion, an interface of different materials, or a combination thereof.

Generally, the one or more microneedles of the microneedle particles may include any structural feature that may assist with [1] the at least partial penetration of a biological tissue, [2] the treatment of a biological tissue, [3] reducing the likelihood of or preventing the removal of the one or more microneedles from a biological tissue, [4] the delivery of the microneedle particles to a particular biological tissue, [5] the fracturing of the microneedle particles, [6] the delivery of a substance of interest, or a [7] a combination thereof. For example, each of the one or more microneedles of the microneedle particles independently may be barbed, curved, perforated, hollow, pocketed, swellable/expandable, or a combination thereof.

The one or more microneedles may be configured to reduce or eliminate the microneedles' ability to at least partially penetrate a biological tissue after use. In one embodiment, upon penetrating a biological tissue at least once, at least one of the one or more microneedles are configured to fail mechanically, thereby preventing the at least one of the one or more microneedles from re-penetrating the biological tissue. In another embodiment, upon penetrating a biological tissue at least once, at least one of the one or more microneedles are configured to fail chemically, thereby preventing the at least one of the one or more microneedles from re-penetrating the biological tissue. In yet another embodiment, upon penetrating a biological tissue at least once, at least one of the one or more microneedles are configured to fail mechanically and chemically, thereby preventing the at least one of the one or more microneedles from re-penetrating the biological tissue. The mechanical and/or chemical failures may occur after the one or more microneedles penetrate a biological tissue once, twice, three times, or more. Non-limiting examples of mechanical failures include damaging the microneedles to change their shape and/or dull their tip. Non-limiting examples of chemical failures include at least partially dissolving a portion of the microneedles, thereby dulling and/or causing changes to the microneedle shape and/or mechanical properties.

The microneedle particles may be made of one or more biocompatible materials, such as metals, polymers, ceramics, bioactive agents, sugars, sugar alcohols, or a combination thereof. The bioactive agents generally may include one or more drugs, one or more sensors, one or more cosmeceuticals, or a combination thereof. Therefore, the microneedle particles may be made of a combination of bioactive components (drugs, sensors, cosmeceuticals, or a combination thereof) and inactive components (metals, polymers, ceramics, sugars, etc.). If a portion of the microneedle particle remains in and/or on a biological tissue after removal of the microneedle particle, then the portion of the microneedle particle remaining in and/or on the biological tissue may include at least one bioactive component, at least one inactive component, or a combination thereof.

In one embodiment, the microneedle particle is formed of a substance of interest, such as a bioactive agent. That is, the particle is entirely or substantially constructed of a bioactive substance. “Substantially constructed” as used herein means at least 60% by weight the bioactive substance. In one embodiment, the microneedle particles are made of water-insoluble material(s). In another embodiment, the microneedle particles are made of, or include, at least one water-soluble and/or bioerodible material. When the microneedle particles are made of water-soluble and/or biocrodible material(s), the microneedle particles or a portion thereof may safely degrade if left in a biological tissue, or after disposal. In one example, the microneedle particle has a matrix structure and a substance of interest, such as a bioactive agent, dispersed in the matrix structure. The matrix structure may consist of or include a water-soluble or biocrodible material. Therefore, one or more microneedles of the microneedle particles may be dissolvable, and if left in the skin will dissolve within the interstitial fluid, and may release a bioactive agent and/or other substance of interest into the skin if configured to do so. As used herein, the term “biocrodible” means that the structure/material degrades in vivo by dissolution, enzymatic hydrolysis, crosion, resorption, or a combination thereof. In a preferred embodiment, the substance of interest and a matrix material in which the substance of interest is dispersed form the structure of the microneedle particle. In a preferred embodiment, the matrix material of the biocrodible microneedle particle is water soluble, such that the entire microneedle particle dissolves in vivo. In another embodiment, the matrix material of the biocrodible microneedle particle is biodegradable, such that the microneedle particles are not soluble in the form originally inserted into the biological tissue, but undergo a chemical change in the body (e.g., break chemical bonds of a polymer) that renders the products of the chemical change (e.g., monomers or oligomers of the polymer) that are water soluble or otherwise can be cleared from the body.

In one embodiment, the microneedle particle is a metal microneedle particle. A metal microneedle particle is one in which the entire structure of the microneedle particle is made of metal. In another embodiment, the microncedle particle is a polymeric microneedle particle. A polymeric microneedle particle is one in which the entire structure of the microneedle particle is made of one or more polymeric materials. In yet another embodiment, the microneedle particle has a structure that is formed of at least one metal and at least one polymeric material. When the microneedle particle includes a polymeric material, the microneedle particle may have a structure comprising a polymeric matrix and a substance of interest dispersed in the polymeric matrix. The substance of interest, such as a bioactive agent, may be at least substantially evenly dispersed or unevenly dispersed in the polymeric matrix. A bioactive agent dispersed in the polymeric matrix may include one or more drugs, one or more sensors, one or more cosmeceuticals, or a combination thereof. The substance of interest may be dispersed in at least a portion of the polymeric matrix that forms or is part of the core structure, at least one of the one or more microneedles, the tip of at least one of the one or more microneedles, the barb of at least one of the one or more microneedles, the non-barbed portion of at least one of the one or more microneedles, or a combination thereof. The non-barbed portion of a microneedle may be the portion of the microneedle that remains attached to the core structure if the barb dissolves or is separated from the microneedle particle. A substance of interest also may be encapsulated, i.e., disposed within porosities and/or voids in the microneedle particle, by any techniques known in the art, such as those of U.S. Pat. Nos. 7,918,814 and 8,257,324, each of which is incorporated herein by reference.

At least a portion of a substance of interest may be released from a microneedle particle [1] before, while, and/or after the microneedle particle has at least partially penetrated a biological tissue, [2] while and/or after the microneedle particle is actively applied to a biological tissue, [3] while the microneedle particle is in contact with a biological tissue, [4] upon and/or after the removal of the microneedle particle from the biological tissue when a portion of the microneedle particle remains on and/or in the biological tissue, or [5] a combination thereof.

At least a portion of a substance of interest may be released from a microneedle particle by one or more mechanisms, including, but not limited to, [1] diffusion through a portion of the microneedle particle, [2] dissolution into a biological tissue, [3] mechanical separation from the microneedle particle (e.g., peeling, breaking, or crumbling off), [4] cleavage of a covalent and/or non-covalent bond (e.g., hydrolytic or enzymatic bond cleavage), [5] cleavage of a physicochemical force (e.g., change in electrostatic interactions due to pH change), [6] swelling/des welling of a material of which at least a portion of the microneedle particle is formed (e.g., a gel), [7] a phase change of a material of which at least a portion of the microneedle particle is formed (e.g., melting, due, for example, to a temperature change), or [8] a combination thereof.

The microneedle particles provided herein may be made by any suitable method capable of forming a desired geometric shape of the microneedle particles. Non-limiting examples of such methods include molding, mechanical or chemical etching, laser cutting, 3D printing, or other microfabrication techniques known in the art. For example, the microneedle particles may be formed by laser etching a sheet of a material. As a further example, the microneedle particles may be made using a molding process that may include placing a material of construction in a mold having cavities that correspond to the desired geometry of the resulting microneedle particles. The material of construction may be a polymer or precursor thereof, and may be loaded into the mold in a powder or liquid form (e.g., molten polymer and/or polymer dissolved or dispersed in a liquid medium), and then solidified into solid monolithic form in the mold. In another example, an array of discrete particles is formed from a solid sheet of the material by a process that includes at least one of etching, punching, or cutting, such as laser cutting. The microneedle particles also may be sintered. Not wishing to be bound by any particular theory, it is believed that sintering may sharpen the edges and/or tips of the microneedle particles.

In embodiments, the microneedle particle is at least partially coated with a coating composition comprising a bioactive agent and/or other substance of interest. The coating composition, in one embodiment, is applied to the entire surface of the microneedle particle. In another embodiment, the coating composition is applied to at least a portion of the one or more microneedles, the core structure, or a combination thereof. For example, the coating composition may be applied to the core structure, at least one of the one or more microneedles, the tip of at least one of the one or more microneedles, the barb of at least one of the one or more microneedles, or the non-barbed portion of at least one of the one or more microneedles. The coating composition may remain in and/or on the biological tissue upon removal of the microneedle particles. The bioactive agent of the coating composition may include one or more drugs, one or more sensors, one or more cosmeceuticals, or a combination thereof. The coating composition may be applied to the microneedle particles using any technique known in the art, including those of U.S. Patent Application Publication No. 2008/0213461, which is incorporated herein by reference.

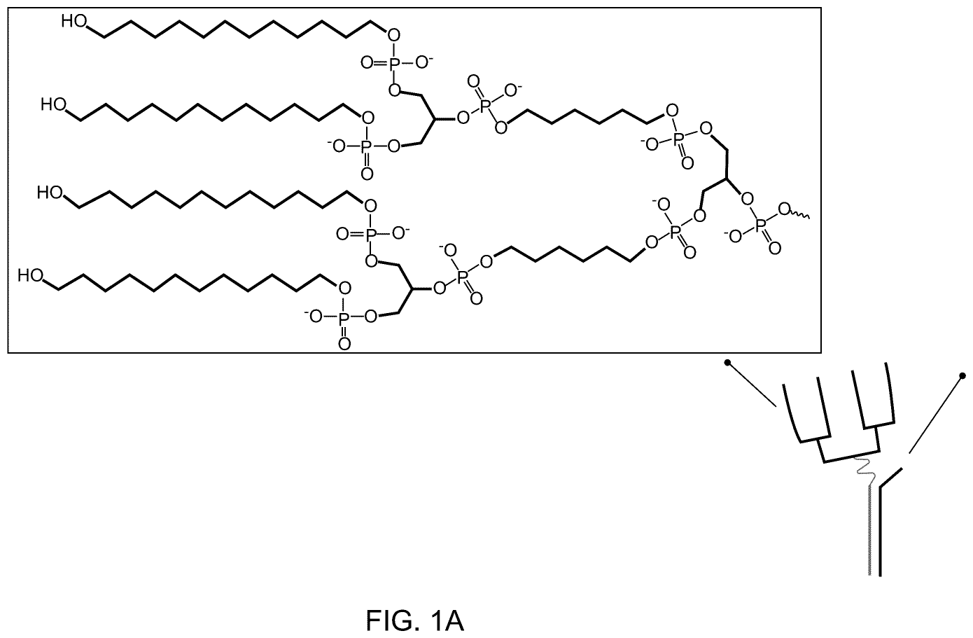

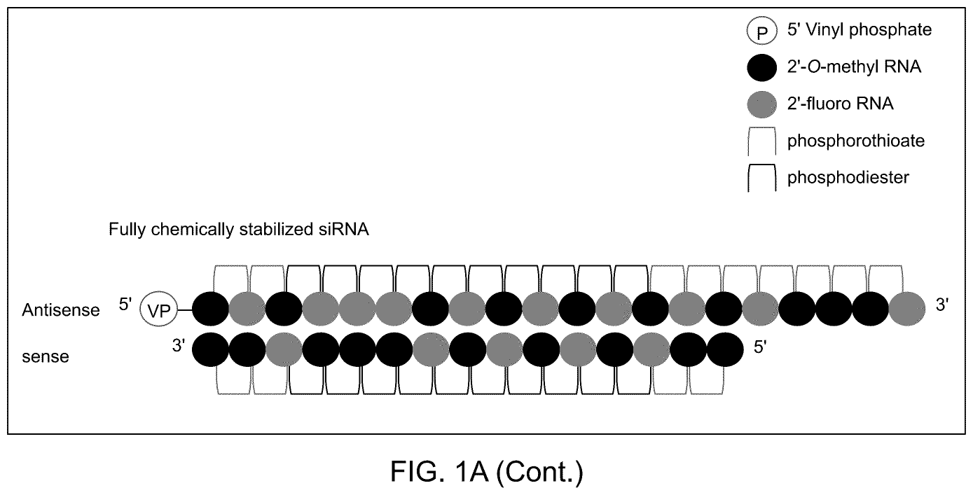

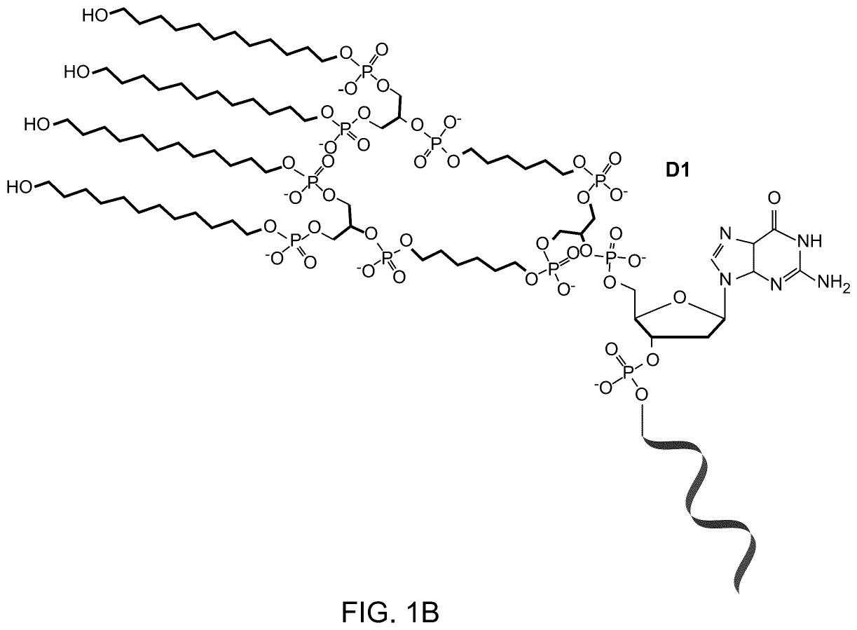

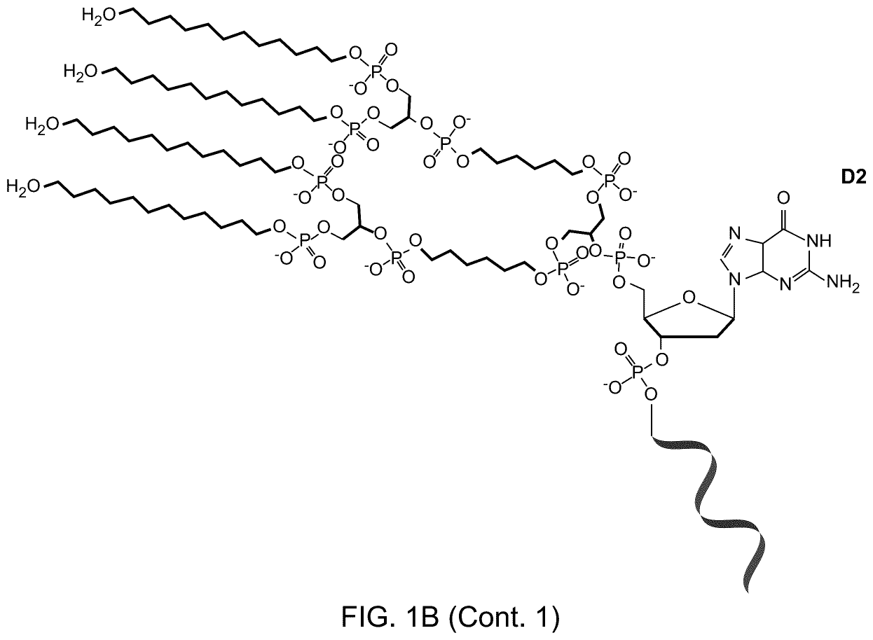

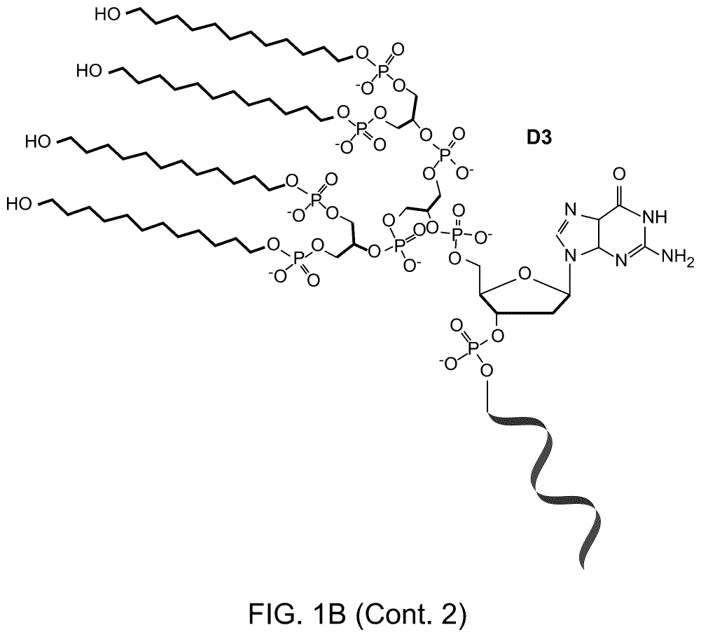

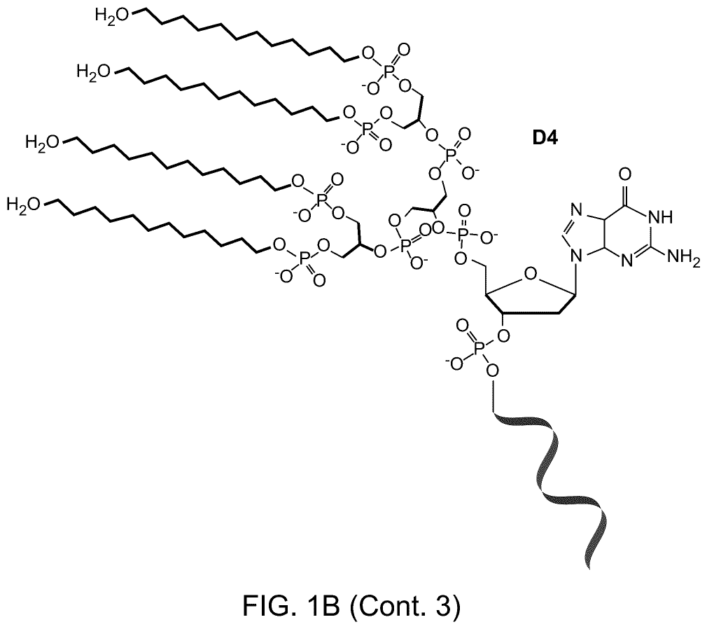

The microneedle particles described herein are described in greater detail in U.S. Pat. No. 11,291,816, the contents of which are incorporated herein by reference. U.S. Pat. No. 11,291,816 provides exemplary schematics of microparticles as well (see FIGS. 1-8 of U.S. Pat. No. 11,291,816).

### EXAMPLES

While several experimental Examples are contemplated, these Examples are intended to be non-limiting.

### Example 1. Oligonucleotide Synthesis

Oligonucleotides were synthesized on a MerMade 6/12 synthesizer (Bioautomation) and AKTA Oligopilot 100 (GE Healthcare Life Sciences) following standard protocols. In brief, conjugated sense strands were synthesized at 5-20 μmol scales on custom-synthesized lipid-functionalized controlled pore glass (CPG) supports for DCA conjugate. For the dendritic sense strand, synthesis was on a CPG functionalized with UnyLinker (ChemGenes) and commercially available amidites (Cy3, C6, C12, and symmetrical branching from ChemGenes and Glen Research) were used to build the dendritic moiety on the 5′-end. All sense strands had a 2dT spacer in between the strand and the conjugate. Antisense strands were synthesized on CPG functionalized with UnyLinker. They were first deprotected with a solution of bromotrimethylsilane/pyridine (3:2, v/v) in dichloromethane for the (E)-vinylphosphonate deprotection, then cleaved and deprotected with 28% aqueous ammonium hydroxide solution for 20 hours at 60° C. All strands were cleaved and deprotected using 28% aqueous ammonium hydroxide solution for 20 hours at 60° C., followed by drying under vacuum at 60° C., and resuspended in Millipore H2O. Oligonucleotides were purified using an Agilent Prostar System (Agilent Technologies) over a C18 column for lipid-conjugated sense strands and over an ion-exchange column for antisense strands. Purified oligonucleotides were desalted by size-exclusion chromatography and characterized by liquid chromatography-mass spectrometry (LC/MS) analysis on an Agilent 6530 accurate-mass quadrupole time-of-flight (Q-TOF) LC/MS (Agilent Technologies). FIG. 1 shows an example of a fully chemically stabilized oligonucleotide (siRNA) and a dendritic (D) siRNA synthesized using this method.

Sequences of compounds and their modifications are shown in Table 1 below (#: PS backbone, m: 2′-o-methyl, f: 2′-fluoro, C12: hexaethylene spacer, C6: triethylene spacer, SB: symmetrical branching, and V: (E)-vinylphosphonate).

TABLE 1
Oligonucleotide sequences synthesized.

| SEQ | | |
|---|---|---|
| ID | | |
| NO | Strands | Sequences (5′ to 3′) |
| 1 | Alkyl-chain | (Cy3)#(mG)#(mU)#(mA)(fC)(mA)(fA)(mA)(fG)(mG)(fA) |
| | hydrophobicity-sense | (mA)(mU)(mC)(fU)#(mG)#(mA)(dT)(dT)-DCA |
| | (skin penetration | (Cy3)#(mG)#(mU)#(mA)(fC)(mA)(fA)(mA)(fG)(mG)(fA) |
| | experiments) | (mA)(mU)(mC)(fU)#(mG)#(mA)(dT)(dT)-EPA |
| 2 | Ringed | (Cy3)#(mC)#(mA)#(mG)(mU)(fA)(fA)(fA)(mG)(fA)(mG) |
| | hydrophobicity-sense | (mA)(mU)(mU)#(mA)#(mA)(dT)(dT)-Chol |
| | (skin penetration | |
| | experiments) | |
| 3 | Large size-sense | (Cy3)#(mC)#(mA)#(mG)(mU)(fA)(fA)(fA)(mG)(fA)(mG) |
| | (skin penetration | (mA)(mU)(mU)#(mA)#(mA)-Diol |
| | experiments) | |
| 4 | Albumin binding-sense | (C12)(SB)(C6)(SB)(dT)(dT)(Cy3)#(mG)#(mU)#(mA)(fC) |
| | (skin penetration | (mA)(fA)(mA)(fG)(mG)(fA)(mA)(mU)(mC)(fU)#(mG)#(mA) |
| | experiments) | |
| 5 | Antisense (skin | V(mU)#(fC)#(mA)(fG)(fA)(fU)(mU)(fC)(mC)(fU)(mU) |
| | penetration | (fU)(mG)(fU)#(mA)#(fC)#(mU)#(mU)#(mC)#(fA)#(mU) |
| | experiments) | |
| 6 | Dendritic (D)-sense | (C12)(SB)(C6)(SB)(dT)(dT)(mG)#(mU)#(mA)(fC)(mA) |
| | (JAK1 targeting) | (fA)(mA)(fG)(mG)(fA)(mA)(mU)(mC)(fU)#(mG)#(mA) |
| 7 | Unc-sense (JAK1 | (mG)#(mU)#(mA)(fC)(mA)(fA)(mA)(fG)(mG)(fA)(mA) |
| | targeting) | (mU)(mC)(fU)#(mG)#(mA)(dT)(dT) |
| 8 | DCA-sense (JAK1 | (mG)#(mU)#(mA)(fC)(mA)(fA)(mA)(fG)(mG)(fA)(mA) |
| | targeting) | (mU)(mC)(fU)#(mG)#(mA)(dT)(dT)-DCAv1 |
| 9 | Antisense (JAK1 | V(mU)#(fC)#(mA)(fG)(fA)(fU)(mU)(fC)(mC)(fU)(mU) |
| | targeting) | (fU)(mG)(fU)#(mA)#(fC)#(mU)#(mU)#(mC)#(fA)#(mU) |
| 10 | DCA NTC-sense | (mA)#(mU)#(mU)(fG)(mA)(fC)(mA)(fA)(mA)(fU)(mA) |
| | (JAK1 experiments) | (mC)(mG)(fA)#(mU)#(mA)(dT)(dT)-DCAv1 |
| 11 | NTC-antisense (JAK1 | V(mU)#(fA)#(mU)(fC)(fG)(fU)(mA)(fU)(mU)(fU)(mG) |
| | experiments) | (fU)(mC)(fA)#(mA)#(fU)#(mC)#(mU)#(mU)#(fU)#(mU) |

### Example 2. Lipoprotein Size Exclusion Chromatography

For lipoprotein profiling, the same protocol previously described by Osborne was followed (see Osborn, M. F. et al. “Hydrophobicity drives the systemic distribution of lipid-conjugated siRNAs via lipid transport pathways,” Nucleic Acids Research 47, 1070-1081 (2018), incorporated herein by reference in their entirety). Briefly, mice were injected intravenously with 10 mg/kg of Cy3-labeled oligonucleotides. After 15 minutes, whole mouse blood (˜500 μl) was collected in a sterile EDTA-coated tube following check incision with a lancet. Samples were spun at 10,000 RPM for 10 minutes at 4° C. 50 μl of plasma was directly injected on Superose 6 Increase 10/300 size exclusion column (GE Healthcare). Oligonucleotide migration was monitored at 570 nm, and lipoprotein protein content was monitored by absorbance at 280 nm. For subcutaneous injections, collection was performed at 1 hour post injection.

### Example 3. Reverse Phase HPLC Analysis Of D-siRNA vs DCA-siRNA

The LC data of oligonucleotides was performed on an Agilent 6530 accurate mass Q-TOF using the following conditions: buffer A: 100 mM 1,1,1,3,3,3-hexafluoroisopropanol (HFIP) and 9 mM triethylamine (TEA) in LC-MS grade water; buffer B: 100 mM HFIP and 9 mM TEA in LC-MS grade methanol; column, Agilent AdvanceBio oligonucleotides C18; 5-100% B 11 min; temperature, 60° C.; flow rate, 0.5 ml/min. LC peaks were monitored at 260 nm.

### Example 4. D-siRNA's Selective Protein Binding Profile to Albumin

A lipoprotein binding profile via size exclusion chromatography, as previously developed by Osborne, was assessed (see Osborn, M. F. et al. “Hydrophobicity drives the systemic distribution of lipid-conjugated siRNAs via lipid transport pathways,” Nucleic Acids Research 47, 1070-1081 (2018), incorporated herein in their entirety).

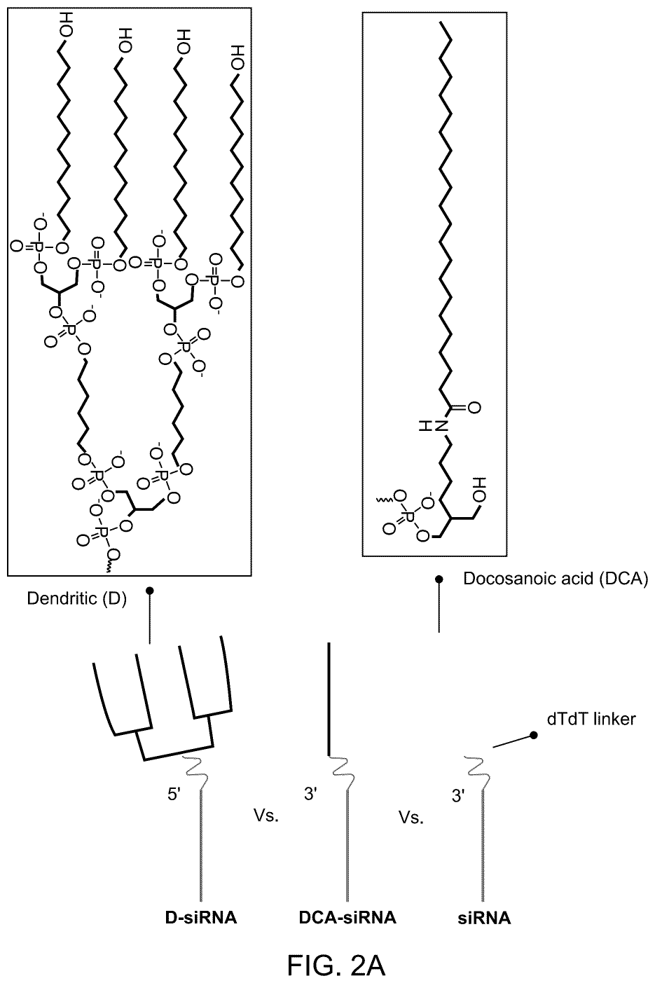

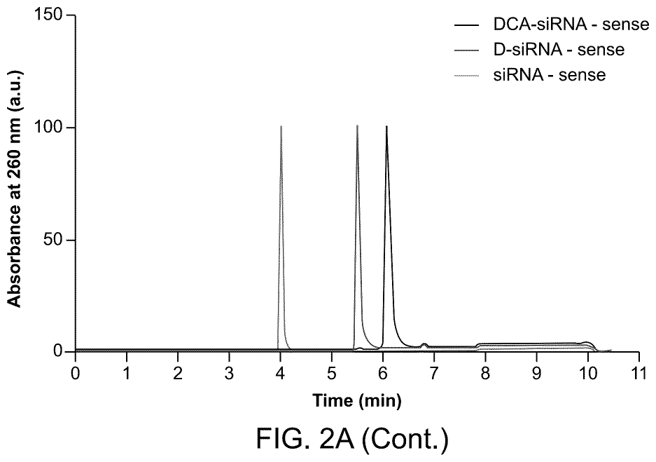

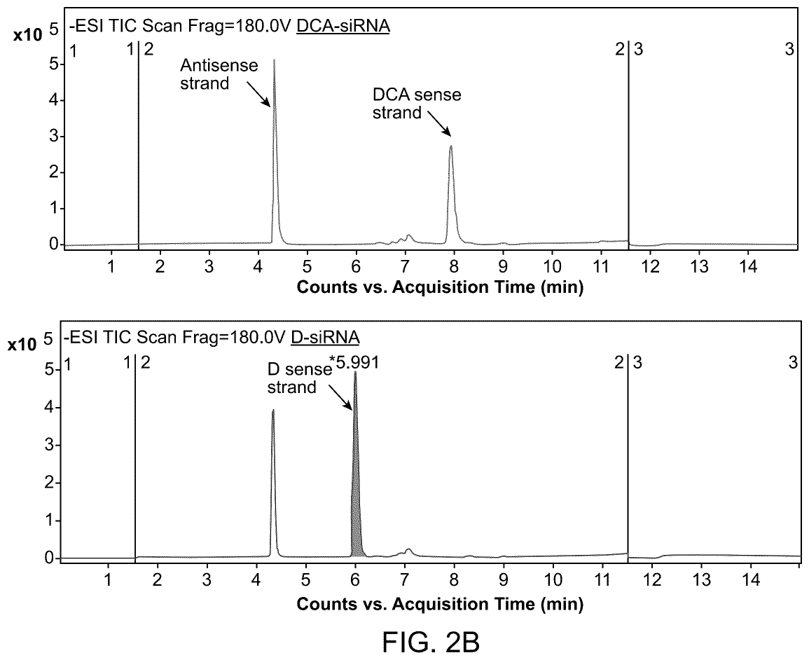

The binding profile of a conjugate partly depends on its hydrophobicity, where more hydrophobic conjugates (such as docosanoic acid, DCA) bind to low-density and high-density lipoproteins (LDL and HDL) in plasma, and to a lesser extent with albumin. The dendritic moicty is larger and has more aliphatic material (69 carbons) compared to DCA (28 carbons). The multiple phosphates that punctuate the structure of the conjugate, in addition to the terminal hydroxyl group, can lead to an increased solubility and prevention of aggregation. Consequently D-siRNA is less hydrophobic than DCA-siRNA, which is evident from lower retention when analyzed by reverse-phase high-performance liquid chromatography (RP-HPLC) (FIG. 2A and FIG. 2B).

Following the hydrophobicity comparison by HPLC, the serum protein binding profile of each conjugate post-injection in mice was examine as previously described. Briefly, animals were injected either subcutaneously (s.c.) or intravenously (i.v.) with Cy3-labeled D-siRNA or DCA-siRNA at a dose of 10 mg/kg, and plasma was collected 15 mins (for i.v.) and 60 minutes (for s.c.) post-injection. The timepoint were chosen to maximize the compounds circulating levels in the blood.

The plasma was then fractionated by size exclusion chromatography, and the Cy-3-siRNA elution time was monitored (570 nm). The Sucrose 6 clusion profile for multiple major plasma proteins (HDL, VLDL, LDL, albumin/globulins) was pre-established. D-siRNA following injection into mice has a retention time of 67 mins, which overlaps with that of albumin, compared to DCA-siRNA which has a multiprotein binding profile associated mostly with LDL and HDL (Error! Reference source not found.B). The protein binding trend was consistent in both routes of administration. This data confirms the selective and high-affinity binding to albumin that is hypothesized to dictate its behavior in vivo as it did in vitro. The data also suggests that D-siRNAs can bind tightly to albumin and circulate as a protein-RNA complex in the blood until it extravasates into various organs and tissues. This is regardless of the mode of injection (subcutaneously or intravenously).

### Example 5. Topical Delivery of Oligonucleotide Conjugates and Branched Oligonucleotides

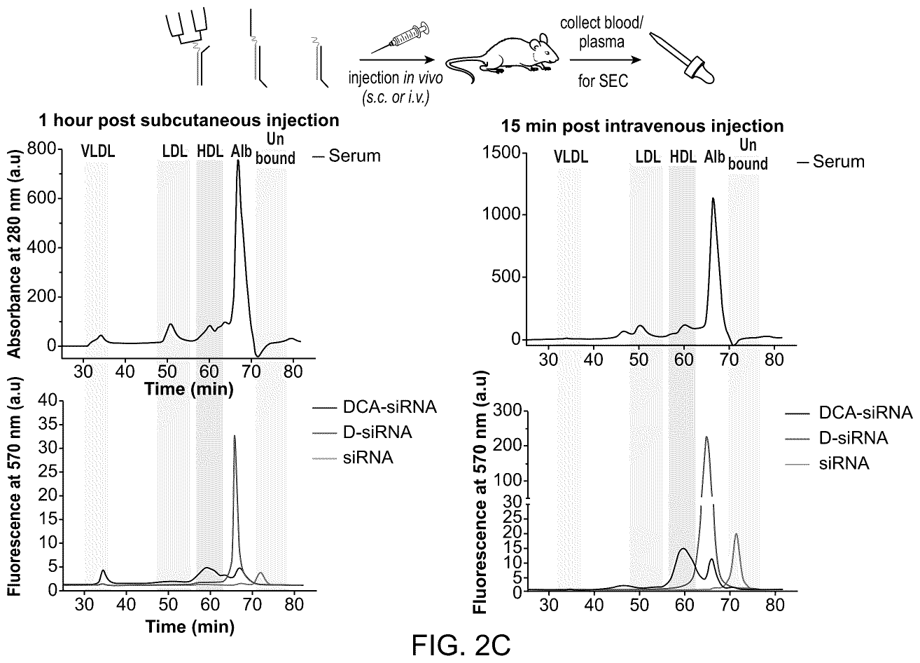

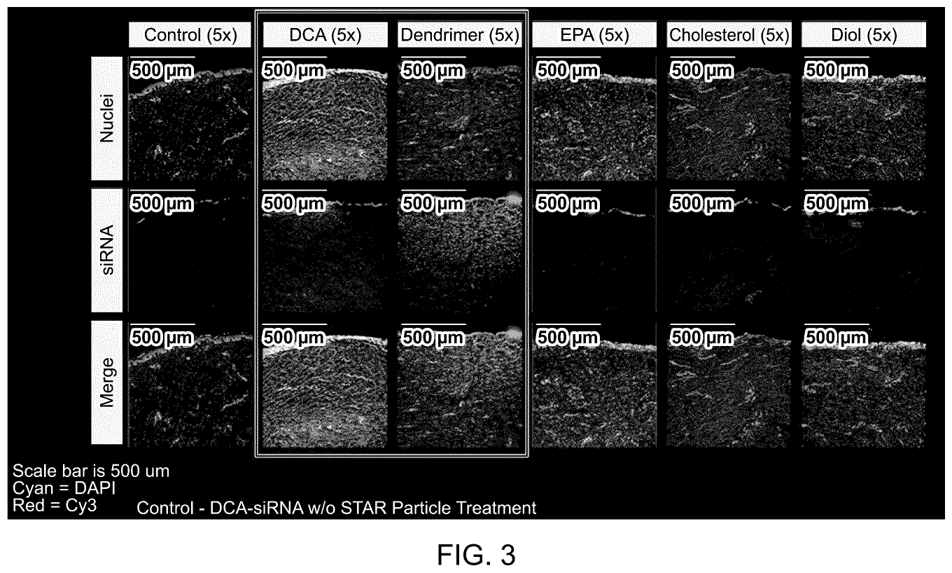

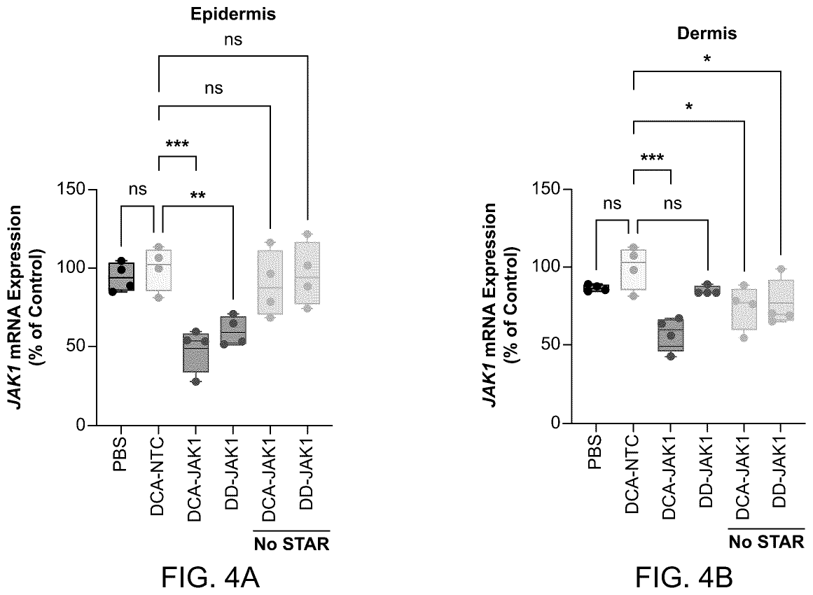

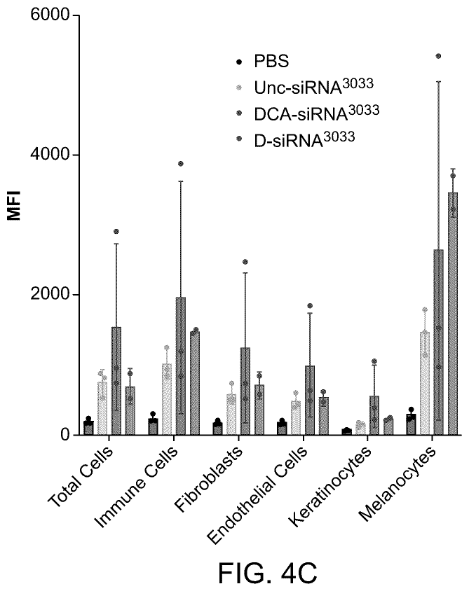

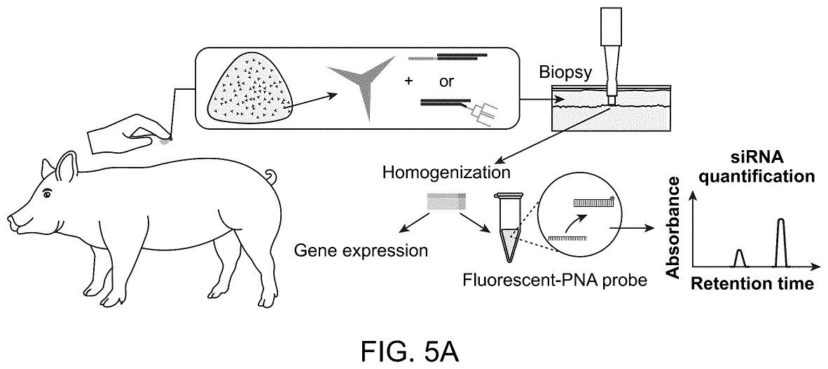

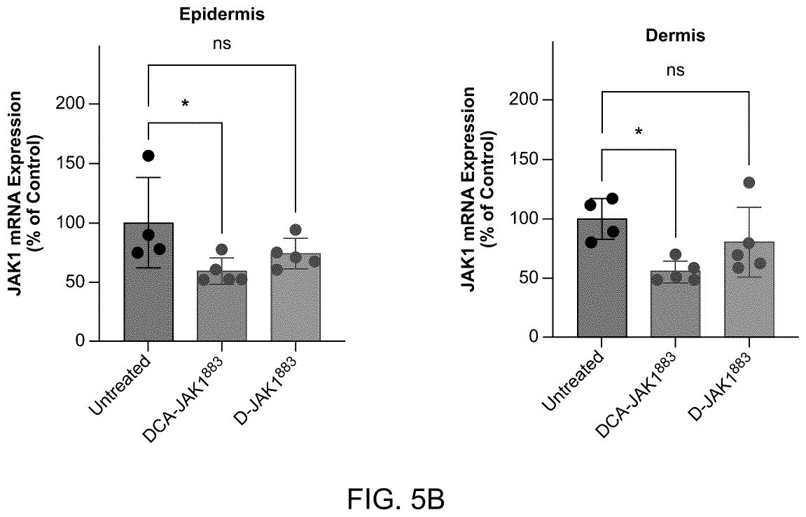

The topical delivery of oligonucleotide conjugates and branched oligonucleotides was assessed (FIG. 3, FIG. 4, and FIG. 5).

For topical delivery, human skin pannus was obtained after surgery. The skin was then cleaned from fat and maintained hydrated. Skin was then rubbed with a mixture of Aloe vera gel and star particle microneedles to create punctures. Rubbing was from 5-15 mins, followed by removal of microneedles and aloe vera gel. Then, Cy3-labeled siRNAs with various conjugates were applied topically to the region treated with the microneedles and left to incubate 24 hours overnight in culture media (175 μM in 240 μL). Fluorescent microscopy images were then obtained to track the delivery of the various siRNA conjugates into the skin layers (epidermis, dermis etc.) As shown in FIG. 3, all conjugates (DCA, EPA, cholesterol, and the dendrimer) and the di-branched oligonucleotide (diol) showed effective retention in the skin following the microneedle particle treatment. The DCA-conjugated oligonucleotide showed the best retention, while the dendrimer produced a more diffuse and deeper spread.

Gene expression of an exemplary target (i.e., JAK1) was measured in human skin samples prepared as described above. A 1-minute Star particle treatment with about 40 kPa pressure was applied to the skin sample. An 8 mm punch was taken and incubated with 200 μL (200 μM) of the DCA or dendrimer conjugate, 96-hour incubation. As shown in FIG. 4A and FIG. 4B, the DCA and dendrimer conjugated oligonucleotides triggered robust JAK1 silencing in the microneedle-treated skin samples and not in the untreated skin samples. This silencing was observed in the epidermis and dermis. Dendrimer conjugate-based silencing in the dermis was low, but this was attributed to the diffusion effect of the dendrimer and the size of the punch taken. As shown in FIG. 4C, higher cell distribution was observed for conjugated siRNA.

JAK1 mRNA expression levels were also measured in skin samples treated with a siRNA formulation comprising STAR particles. The assay was performed with a composition comprising about 3.5 mg (270 nmols) of siRNA formulated with STAR particles and excipient for a total of 225 mg. The skin sample was rubbed for about 60 seconds on a 3×3 cm zone and the analysis was performed after 4 days of application (FIG. 5A). As shown in FIG. 5B, the DCA and dendrimer conjugated oligonucleotides triggered robust JAK1 silencing.

Quantification of JAK1 mRNA was measured using Quantagene singleplex assay. Data (JAK1 mRNA) is expressed as a percentage of a non-targeting control.

### INCORPORATION BY REFERENCE

The contents of all cited references (including literature references, patents, patent applications, patent publications, and websites) that maybe cited throughout this application are hereby expressly incorporated by reference in their entirety for any purpose, as are the references cited therein. The disclosure will employ, unless otherwise indicated, conventional techniques of immunology, molecular biology and cell biology, which are well known in the art.

The present disclosure also incorporates by reference in their entirety techniques well known in the field of molecular biology and drug delivery. These techniques include, but are not limited to, techniques described in the following publications:
Nguyen, V. H. & Lee, B.-J. Protein corona: a new approach for nanomedicine design. Int J Nanomedicine 12, 3137-3151 (2017).
Banker, M. J. & Clark, T. H. Plasma/serum protein binding determinations. Curr Drug Metab 9, 854-859 (2008).
Zhao, Z., Ukidve, A., Krishnan, V. & Mitragotri, S. Effect of physicochemical and surface properties on in vivo fate of drug nanocarriers. Advanced Drug Delivery Reviews 143, 3-21 (2019).
Albanese, A., Tang, P. S. & Chan, W. C. W. The Effect of Nanoparticle Size, Shape, and Surface Chemistry on Biological Systems. Annual Review of Biomedical Engineering 14, 1-16 (2012).
de Castro, C. E. et al. The Protein Corona Conundrum: Exploring the Advantages and Drawbacks of its Presence around Amphiphilic Nanoparticles. Bioconjugate Chemistry 31, 2638-2647 (2020).
Eckstein, F. Phosphorothioates, essential components of therapeutic oligonucleotides. Nucleic Acid Ther 24, 374-387 (2014).
Agarwal, S. et al. Impact of Serum Proteins on the Uptake and RNAi Activity of GalNAc-Conjugated siRNAs. Nucleic Acid Therapeutics 31, 309-315 (2021).
Francia, V., Schiffelers, R. M., Cullis, P. R. & Witzigmann, D. The Biomolecular Corona of Lipid Nanoparticles for Gene Therapy. Bioconjugate Chemistry 31, 2046-2059
Osborn, M. F. et al. Hydrophobicity drives the systemic distribution of lipid-conjugated siRNAs via lipid transport pathways. Nucleic Acids Res 47, 1070-1081 (2019).
Kratz, F. Albumin as a drug carrier: design of prodrugs, drug conjugates and nanoparticles. J Control Release 132, 171-183 (2008).
Hoogenboezem, E. N. & Duvall, C. L. Harnessing albumin as a carrier for cancer therapies. Advanced Drug Delivery Reviews 130, 73-89 (2018).
Andersen, J. T. & Sandlie, I. The versatile MHC class I-related FcRn protects IgG and albumin from degradation: implications for development of new diagnostics and therapeutics. Drug metabolism and pharmacokinetics 24, 318-332 (2009).
Schnitzer, J. E. gp60 is an albumin-binding glycoprotein expressed by continuous endothelium involved in albumin transcytosis. American Journal of Physiology-Heart and Circulatory Physiology 262, H246-H254 (1992).
Merlot, A. M., Kalinowski, D. S. & Richardson, D. R. Unraveling the mysteries of serum albumin-more than just a serum protein. Front Physiol 5, 299-299 (2014).
Sarett, S. M. et al. Lipophilic siRNA targets albumin in situ and promotes bioavailability, tumor penetration, and carrier-free gene silencing. Proceedings of the National Academy of Sciences 114, E6490-E6497 (2017).
Liu, H. et al. Structure-based programming of lymph-node targeting in molecular vaccines. Nature 507, 519-522 (2014).
Lau, S. et al. Enhanced Extravasation, Stability and in Vivo Cardiac Gene Silencing via in Situ siRNA-Albumin Conjugation. Molecular Pharmaceutics 9, 71-80 (2012).
Lacroix, A., Edwardson, T. G. W., Hancock, M. A., Dore, M. D. & Sleiman, H. F. Development of DNA Nanostructures for High-Affinity Binding to Human Serum Albumin. Journal of the American Chemical Society 139, 7355-7362 (2017).
Purdie, L., Alexander, C., Spain, S. G. & Magnusson, J. P. Alkyl-Modified Oligonucleotides as Intercalating Vehicles for Doxorubicin Uptake via Albumin Binding. Molecular Pharmaceutics 15, 437-446 (2018).
Bern, M., Sand, K. M. K., Nilsen, J., Sandlie, I. & Andersen, J. T. The role of albumin receptors in regulation of albumin homeostasis: Implications for drug delivery. Journal of Controlled Release 211, 144-162 (2015).
Felber, A. E. et al. The interactions of amphiphilic antisense oligonucleotides with serum proteins and their effects on in vitro silencing activity. Biomaterials 33, 5955-5965 (2012).
Monopoli, M. P., Åberg, C., Salvati, A. & Dawson, K. A. Biomolecular coronas provide the biological identity of nanosized materials. Nature Nanotechnology 7, 779-786 (2012).
Lacroix, A., Fakih, H. H. & Sleiman, H. F. Detailed cellular assessment of albumin-bound oligonucleotides: Increased stability and lower non-specific cell uptake. Journal of Controlled Release 324, 34-46 (2020).
Biscans, A. et al. Docosanoic acid conjugation to siRNA enables functional and safe delivery to skeletal and cardiac muscles. Molecular Therapy 29 (2020).
Biscans, A. et al. Docosanoic acid conjugation to siRNA enables functional and safe delivery to skeletal and cardiac muscles. Molecular Therapy 29, 1382-1394 (2021).
Osborn, M. F. et al. Hydrophobicity drives the systemic distribution of lipid-conjugated siRNAs via lipid transport pathways. Nucleic Acids Research 47, 1070-1081 (2018).
Biscans, A. et al. Diverse lipid conjugates for functional extra-hepatic siRNA delivery in vivo. Nucleic Acids Research 47, 1082-1096 (2018).
A High-Throughput Method for Direct Detection of Therapeutic Oligonucleotide-Induced Gene Silencing In Vivo. Nucleic Acid Therapeutics 26, 86-92 (2016).
Godinho, B. M. D. C. et al. Pharmacokinetic Profiling of Conjugated Therapeutic Oligonucleotides: A High-Throughput Method Based Upon Serial Blood Microsampling Coupled to Peptide Nucleic Acid Hybridization Assay. Nucleic Acid Therapeutics 27 (2017).
Godinho, B. et al. Pharmacokinetic Profiling of Conjugated Therapeutic Oligonucleotides: A High-Throughput Method Based Upon Serial Blood Microsampling Coupled to Peptide Nucleic Acid Hybridization Assay. Nucleic Acid Ther 27, 323-334 (2017).
Sand, K. M. K. et al. Unraveling the Interaction between FcRn and Albumin: Opportunities for Design of Albumin-Based Therapeutics. Frontiers in Immunology 5 (2015).
Lomis, N. et al. Albumin Nanoparticle Formulation for Heart-Targeted Drug Delivery: In Vivo Assessment of Congestive Heart Failure. Pharmaceuticals (Basel) 14 (2021).
John, T. A. et al. Evidence for the role of alveolar epithelial gp60 in active transalveolar albumin transport in the rat lung. The Journal of Physiology 533, 547-559 (2001).
Woods, A. et al. In vivo biocompatibility, clearance, and biodistribution of albumin vehicles for pulmonary drug delivery. Journal of controlled release: official journal of the Controlled Release Society 210, 1-9 (2015).
Dheilly, E. et al. Selective Blockade of the Ubiquitous Checkpoint Receptor CD47 Is Enabled by Dual-Targeting Bispecific Antibodies. Mol Ther 25, 523-533 (2017).
Shin, M. et al. Intratracheally administered LNA gapmer antisense oligonucleotides induce robust gene silencing in mouse lung fibroblasts. Nucleic Acids Res 50, 8418-8430 (2022).
Didiot, M. C. et al. Nuclear Localization of Huntingtin mRNA Is Specific to Cells of Neuronal Origin. Cell Rep 24, 2553-2560.e2555 (2018).
Biscans, A. et al. Diverse lipid conjugates for functional extra-hepatic siRNA delivery in vivo. Nucleic acids research 47, 1082-1096 (2019).
Nikan, M. et al. Docosahexaenoic acid conjugation enhances distribution and safety of siRNA upon local administration in mouse brain. Molecular Therapy-Nucleic Acids 5, e344 (2016).
Alterman, J. F. et al. Hydrophobically modified siRNAs silence huntingtin mRNA in primary neurons and mouse brain. Molecular Therapy-Nucleic Acids 4, e266 (2015).
Davis, S. M. et al. Chemical optimization of siRNA for safe and efficient silencing of placental sFLT1. Mol Ther Nucleic Acids 29, 135-149 (2022).
Ly, S., Echeverria, D., Sousa, J. & Khvorova, A. Single-Stranded Phosphorothioated Regions Enhance Cellular Uptake of Cholesterol-Conjugated siRNA but Not Silencing Efficacy. Molecular Therapy-Nucleic Acids 21, 991-1005 (2020).
Malhotra, A. & Mittal, B. R. SiRNA gene therapy using albumin as a carrier. Pharmacogenetics and Genomics 24 (2014).

### EQUIVALENTS

The disclosure may be embodied in other specific forms without departing from the spirit or essential characteristics thereof. The foregoing embodiments are therefore to be considered in all respects illustrative rather than limiting of the disclosure. Scope of the disclosure is thus indicated by the appended claims rather than by the foregoing description, and all changes that come within the meaning and range of equivalency of the claims are therefore intended to be embraced herein.

1. A method for topical delivery of an oligonucleotide conjugate or a branched oligonucleotide to a subject, the method comprising:
a) contacting a region of skin of the subject with a plurality of microneedle particles: and
b) contacting the region of skin from step a) with the oligonucleotide conjugate or the branched oligonucleotide, or
c) contacting a region of skin of the subject with a topical formulation comprising:
a plurality of microneedle particles;
an oligonucleotide conjugate or a branched oligonucleotide;
optionally a pharmaceutically acceptable carrier; and
optionally a pharmaceutically acceptable salt,
wherein the oligonucleotide conjugate comprises:
i) an oligonucleotide comprising a 5′ end, a 3′ end, and complementarity to a target nucleic acid; and
ii) a functional moiety linked to the oligonucleotide.

2. The method of claim 1, wherein each of the plurality of microneedle particles comprises a core structure and one or more microneedles extending from the core structure.

3. The method of claim 2, wherein;
the one or more microneedles is structured to penetrate a biological tissue; or
at least one of (i) the core structure, (ii) the one or more microneedles, and (iii) a spatial relationship between or among two or more of the microneedles is configured to prevent the entire plurality of microneedle particles from penetrating the biological tissue.

4. (canceled)

5. The method of claim 1, wherein each of the plurality of microneedle particles comprises:
a core structure; and
three microneedles, four microneedles, five microneedles, six microneedles, seven microneedles, eight microneedles, or ten microneedles extending from the core structure.

6-7. (canceled)

8. The method of claim 3, wherein each of the one or more microneedles independently has a length of about 10 μm to about 2,000 μm, about 100 μm to about 1,000 μm, or about 100 μm to about 500 μm.

9-11. (canceled)

12. The method of claim 3, wherein:
upon penetrating the biological tissue at least once, at least one of the one or more microneedles is configured to fail mechanically, thereby preventing the at least one of the one or more microneedles from re-penetrating the biological tissue; or

upon penetrating the biological tissue at least once, at least one of the one or more microneedles is configured to fail chemically, thereby preventing the at least one of the one or more microneedles from re-penetrating the biological tissue.

13-18. (canceled)

19. The method of claim 1, wherein the functional moiety comprises any one of a dendron, a retinoic acid, a docosahexaenoic acid (DHA), a docosanoic acid (DCA), and an a-tocopheryl succinate.

20-22. (canceled)

23. The method of claim 19, wherein the dendron comprises two branches, four branches, or eight branches.

24-25. (canceled)

26. The method of claim 1, wherein the oligonucleotide conjugate has a structure of Formula I: 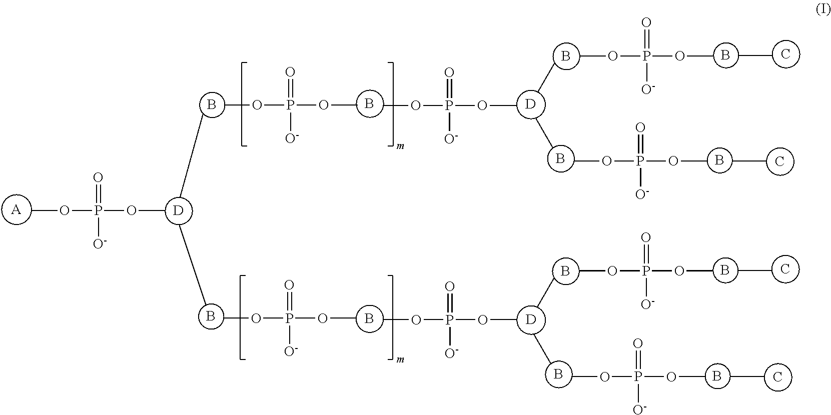
wherein:
A is an oligonucleotide;
B, for each occurrence, independently comprises one or more hydrophobic chains, amines, amides, esters, an N -or O-containing heterocycle, thioethers, disulfides, and/or aromatic rings, wherein the one or more hydrophobic chains comprise a saturated or an unsaturated C₁₋₂₄ alkyl chain;
C, for each occurrence, independently comprises: an hydrophilic group comprising a hydroxide, an amine, a phosphate, a sulfur, and/or a sugar; an hydrophobic group comprising an amine, an amide, an ether, an ester, a N- or O-containing heterocycle, a thiol, a thioether, and/or a saturated or an unsaturated C₁₋₂₄ alkyl chain; and/or one or more aromatic rings;
D, for each occurrence, independently is a branching unit comprising one or more alkyl chains, amides, ethers, esters, and amines, wherein the branching unit comprises 2 to 4 branches; and
m, for each occurrence, independently is 0 or 1.

27. The method of claim 26, wherein:
the oligonucleotide conjugate has a structure of Formula II: 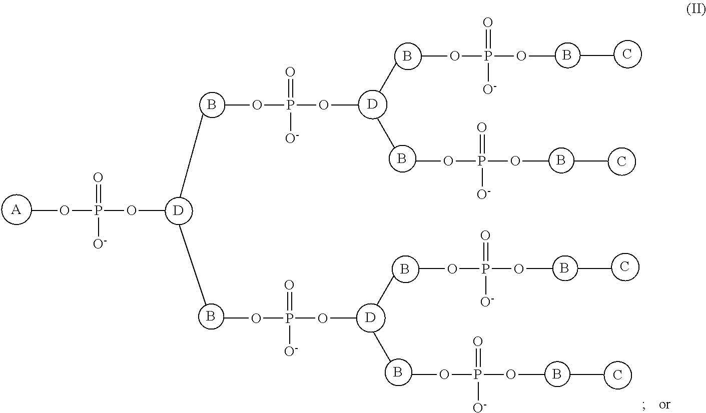
the oligonucleotide conjugate has a structure of Formula III: 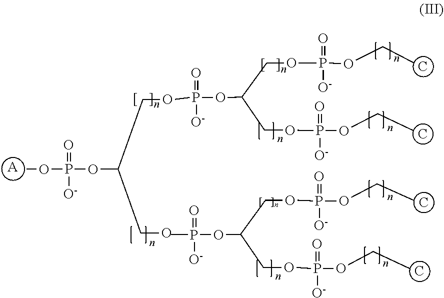
wherein n, for each occurrence, independently is an integer between 1-24; or
C is OH and n, for each occurrence, independently is 1, 6, or 12; or
wherein the oligonucleotide conjugate has the structure of Formula IV: 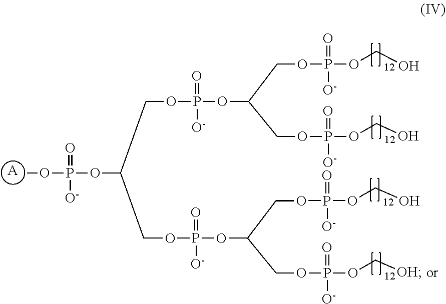
the oligonucleotide conjugate has a structure of Formula V:

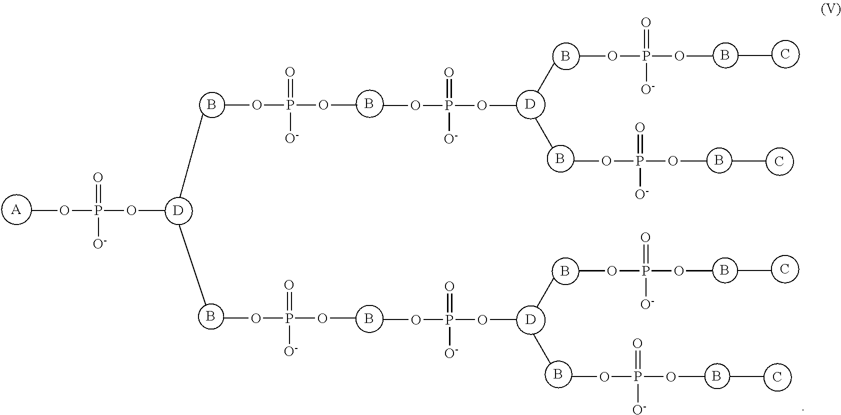

28-31. (canceled)

32. The method of claim 26, wherein:
the oligonucleotide conjugate has a structure of Formula VI: 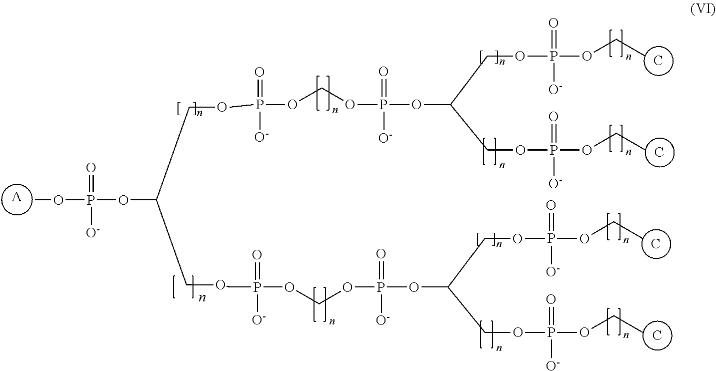
wherein n, for each occurrence, independently is an integer between 1-24, optionally wherein C is OH and n, for each occurrence, independently is 1, 6, or 12, optionally wherein the oligonucleotide conjugate has the structure of Formula VII:

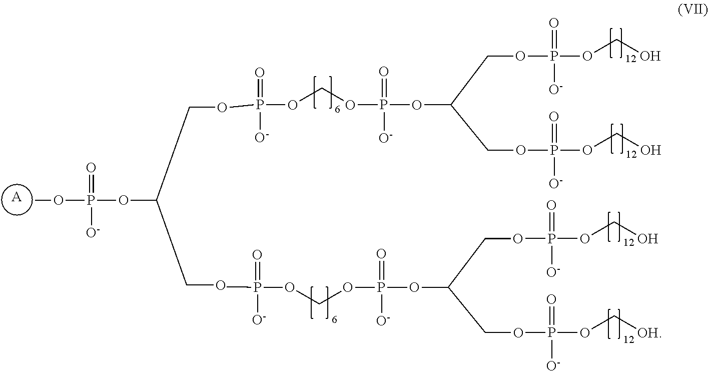

33-35. (canceled)

36. The method of claim 1, wherein the oligonucleotide corresponds to an antisense oligonucleotide or a siRNA.

37. The method of claim 36, wherein the siRNA comprises a sense strand and an antisense strand, each comprising a 5′ end and a 3′ end.

38. The method of claim 37, wherein the functional moiety is linked to the 5′ end and/or the 3′ end of the sense strand or to the 5′ end and/or the 3′ end of the antisense strand.

39. (canceled)

40. The method of claim 1, wherein the functional moiety is linked to the oligonucleotide by a linker.

41-45. (canceled)

46. The method of claim 1, wherein the oligonucleotide conjugate comprises the structure:

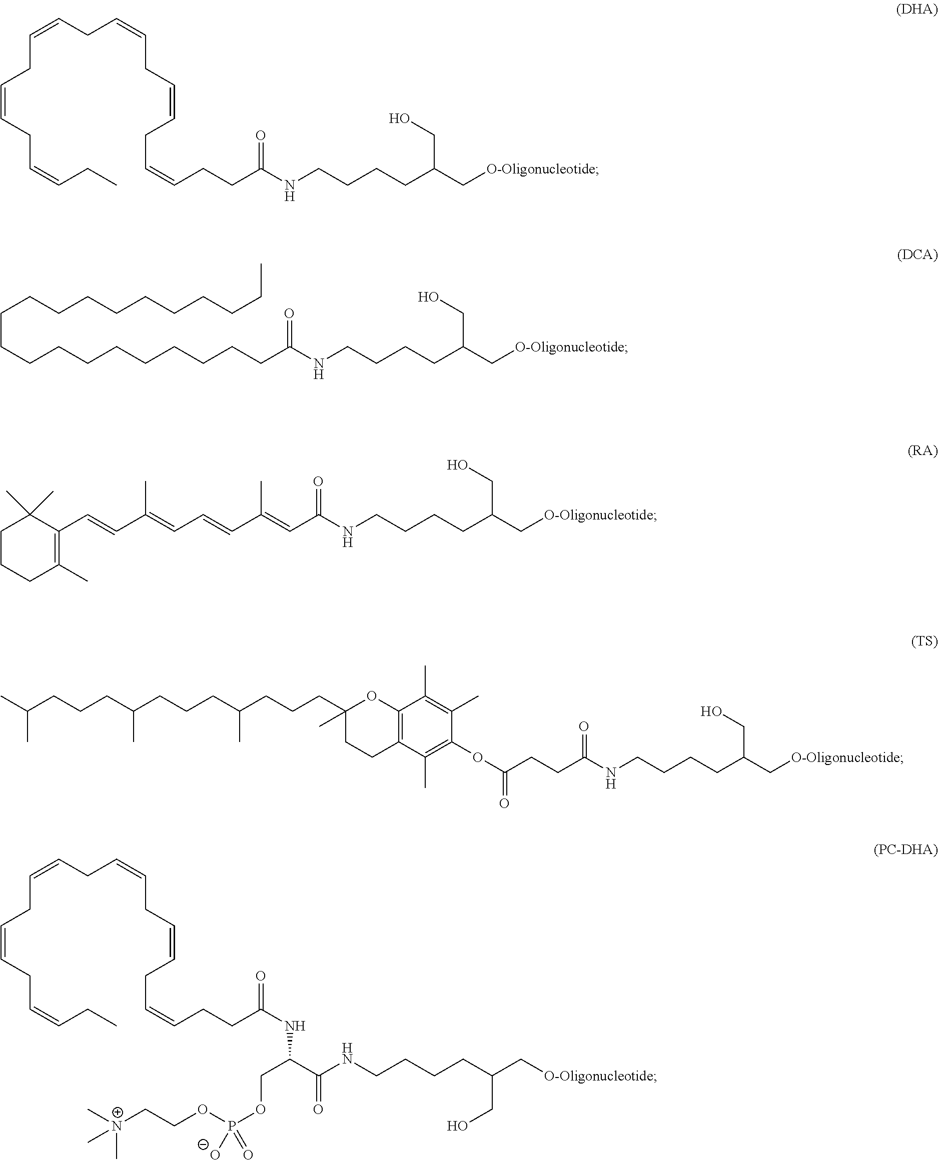

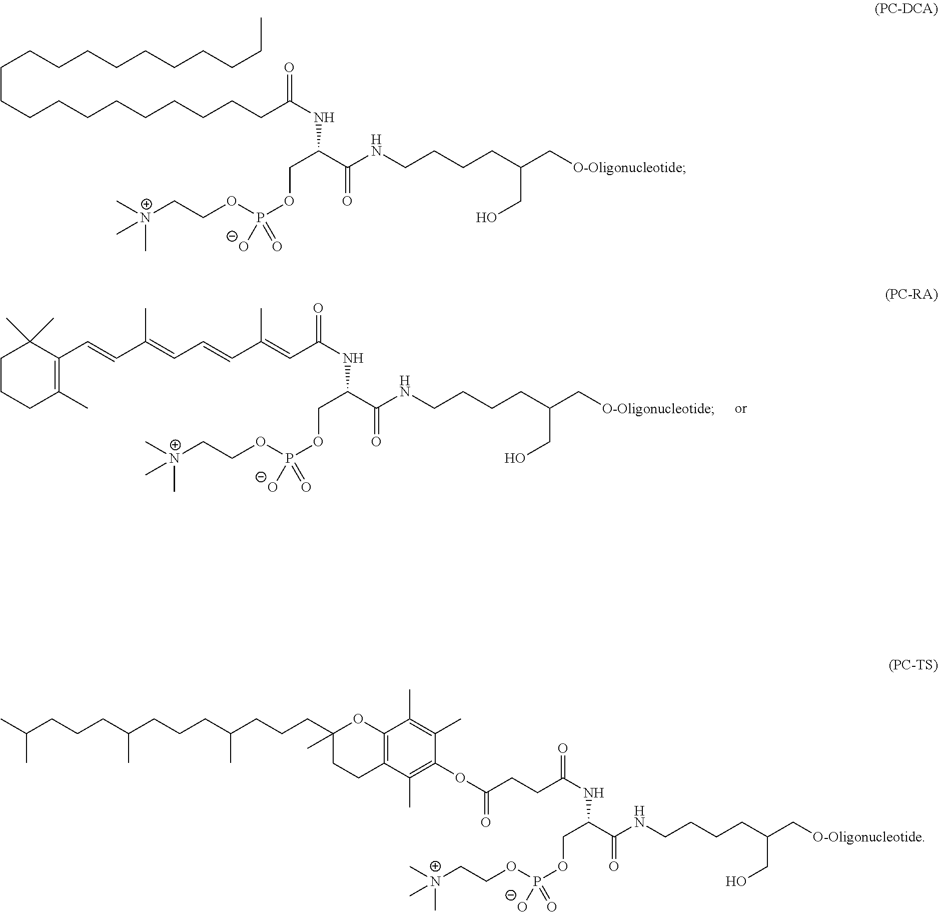

47. The method claim 1, wherein the branched oligonucleotide comprises the structure:

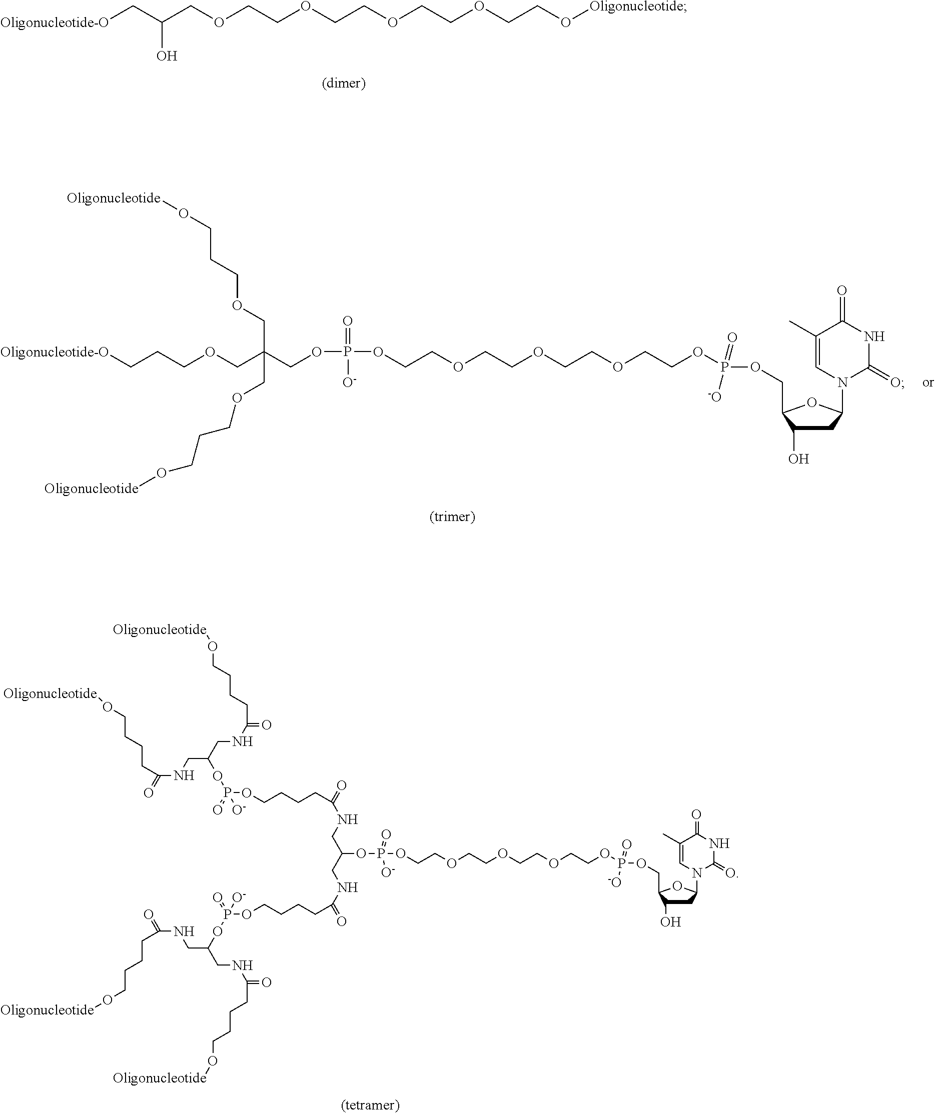

48. The method of claim 37, wherein the antisense strand comprises about 15 nucleotides to about 25 nucleotides in length and/or wherein the sense strand comprises about 15 nucleotides to about 25 nucleotides in length.

49-51. (canceled)

52. The method of claim 37, wherein the siRNA comprises a double-stranded region of about 15 base pairs to about 20 base pairs.

53-56. (canceled)

57. The method of claim 37, wherein the siRNA comprises at least one modified nucleotide.

58. (canceled)

59. The method of claim 37, wherein the siRNA comprises at least one modified internucleotide linkage.

60-68. (canceled)

69. A topical formulation comprising:
(a) a plurality of microneedle particles; and
(b) an oligonucleotide conjugate or branched oligonucleotide, wherein the oligonucleotide conjugate comprises:
i) an oligonucleotide comprising a 5′ end, a 3′ end, and complementarity to a target nucleic acid; and
ii) a functional moiety linked to the oligonucleotide;
(c) optionally a pharmaceutically acceptable carrier; and
(d) optionally a pharmaceutically acceptable salt.
